(19) United States
(12) Patent Application Publication
(10) Pub. No.: US 2002/0164585 A1
(43) Pub. Date: Nov. 7, 2002

(54) Method for enhancing RNA or protein production using non-native 5' untranslated sequences in recombinant viral nucleic acids

(76) Inventors: Sean Chapman, Wormit (GB); William O. Dawson, Winter Haven, FL (US); Jonathan Donson, Oak Park, CA (US); Monto H. Kumagai, Davis, CA (US); Dennis J. Lewandowski, Auburndale, FL (US); John A. Lindbo, Vacaville, CA (US); Gregory P. Pogue, Vacaville, CA (US); Shailaja Shivprasad, Orlando, FL (US)

Correspondence Address: HOWREY SIMON ARNOLD & WHITE, LLP, BOX 34, 301 RAVENSWOOD AVE., MENLO PARK, CA, 94025, US

(21) Appl. No.: 10/057,558

(22) Filed: Jan. 25, 2002

Related U.S. Application Data
(63) Continuation of application No. 09/359,299, filed on Jul. 21, 1999.
Application No. 09/359,299 is a continuation-in-part of application No. 09/232,170, filed on Jan. 15, 1999.
Application No. 09/232,170 is a continuation-in-part of application No. 09/008,186, filed on Jan. 16, 1998.

(51) Int. Cl. A01H 5/00; C07H 21/04

(52) U.S. Cl. 435/5; 536/23.72; 800/288

(57) ABSTRACT

The present invention provides a method for enhancing the production of RNAs or proteins in a plant host using either non-native 5′ untranslated sequences or artificial leader sequences. Preferably, commercially useful proteins, polypeptides, or fusion products thereof are produced, such as, enzymes, antibodies, hormones, pharmaceuticals, vaccines, pigments, anti-microbial polypeptides, and the like. The non-native 5′ untranslated enhancers may also be effective in many different types of transcription or translation systems, such as bacterial and animal systems.

The present Application is a Continuation Application of U.S. Ser. No. 09/359,299 filed Jul. 21, 1999, which is a Continuation-in-Part Application of U.S. Ser. No. 09/232,170 filed Jan. 15, 1999, which is a Continuation-in-Part Application of U.S. Ser. No. 09/008,186 filed Jan. 16, 1998, both of which are incorporated herein by reference.

### FIELD OF INVENTION

This invention relates generally to the field of molecular biology and viral genetics. Specifically, this invention relates to using non-native 5′ untranslated sequences to enhance protein or RNA production by recombinant viral nucleic acids.

### BACKGROUND OF THE INVENTION

Plant proteins and enzymes have long been exploited for many purposes, from viable food sources to biocatalytic reagents, or therapeutic agents. During the past decade, the development of transgenic and transfected plants and improvement in genetic analysis have brought renewed scientific significance and economical incentives to these applications. The concepts of molecular plant breeding and molecular plant farming, wherein a plant system is used as a bioreactor to produce recombinant bioactive materials, have received great attention.

Foreign genes can be expressed in plant hosts either by permanent insertion into the genome or by transient expression using virus-based vectors. Each approach has its own distinct advantages. Transformation for permanent expression needs to be done only once, whereas each generation of plants needs to be inoculated with the transient expression vector. Virus-based expression systems, in which the foreign mRNA is greatly amplified by virus replication, can produce very high levels of proteins in leaves and other tissues. Viral vector-produced protein can also be directed to specific subcellular locations, such as endomembrane, cytosol, or organelles, or it can be attached to macromolecules, such as virions, which aids purification of the protein.

In order for plant-based molecular breeding and farming to gain widespread acceptance in commercial areas, it is necessary to develop methods for increasing the production of bioactive species produced in plants. Factors influencing the production of bioactive species include transcription and translation activities. The mechanisms by which eukaryotes and prokaryotes initiate translation are known to have certain features in common and to differ in others. Eukaryotic messages are functionally monocistronic, translation initiates at the 5′ end and is stimulated by the presence of a cap structure (m⁷G^{5′} ppp5′ G . . . ) at this end (Shatkin, Cell 9:645 (1976)). Prokaryotic messages can be polycistronic, can initiate at sites other than the 5′ terminus, and the presence of a cap does not lead to translational stimulation. Both eukaryotes and prokaryotes begin translation at the codon AUG, although prokaryotes can also use GUG. Translation in both is stimulated by certain sequences near the start codon. For prokaryotes, it is the so-called Shine-Dalgarno sequence (a purine rich region 3-10 nucleotides upstream from the initiation codon). For eukaryotes, it is a purine at the -3 position and a G residue in the+4 position (where the A of the AUG start codon is designated +1), plus other sequence requirements involved in finer tuning. This is part of the “relaxed” version of the scanning model (Kozak, Nuc. Acids. Res. 13:857 (1984)) whereby a 40S ribosomal sub-unit binds at the 5′ end of the eukaryotic mRNA and proceeds to scan the sequence until the first AUG, which meets the requirements of the model, is encountered, at which point a 60S sub-unit joins the 40S sub-unit, eventually resulting in protein synthesis. For sequence requirements related to initiation codon, see publications by Kozak: Cell 15:1109-1123 (1978), Nuc. Acid. Res. 9:5233-5266 (1981) and Cell 44:283-292 (1986).

One of the most widely studied RNA viruses is the Tobacco Mosaic Virus (TMV). Recently, U.S. Pat. No. 5,891,665 issued to Wilson, describes how native 5′ untranslated sequences of TMV, i.e. the omega region, act as enhancers of translation of mRNA. The omega region was previously shown to be related to ribosome association. Shivprasad et al., Virology 255:312-323 (1999) also demonstrated that the presence of a 3′ native nontranslated region affects foreign gene expression in TMV-based vectors.

This invention describes the use of non-native 5′ untranslated sequences to enhance RNA or protein production. Previously, short sequences (4 to 6 base pairs) that mimic the 5′ leader of the coat subgenomic RNA was expected to give optimal expression of foreign genes. For example, the highly expressed TMV-U1 coat subgenomic RNA contains an extremely short 3 bp untranslated leader (AAU). In this invention, the use of non-native sequences at the 5′untranslated region causes an increase in RNA or protein production. These non-native 5′ untranslated sequences act as enhancers of RNA or protein production. Since viral genome is extremely streamlined (Dawson et al., Adv. Virus Res. 38:307-342 (1990)), it is not obvious to include non-native 5′ untranslated sequences in the recombinant viral nucleic acids that will lead to an increase in RNA or protein production.

### SUMMARY OF THE INVENTION

The present invention provides a method for enhancing production of RNAs or proteins in plant hosts using either non-native 5′ untranslated sequences or artificial leader sequences in recombinant viral nucleic acids. These foreign sequences may encode commercially useful proteins, polypeptides, or fusion products thereof, such as enzymes, antibodies, hormones, pharmaceuticals, vaccines, pigments, antimicrobial polypeptides, and the like. These enhancer sequences may be ligated upstream of an appropriate mRNA or used in the form of a cDNA expression vector. The non-native enhancers may also be effective in many different types of transcription or translation systems, such as bacterial and animal systems

### BRIEF DESCRIPTION OF THE FIGURES

FIG. 1. Rice α-amylase expression vector, TTO1A 103L (SEQ ID NOs: 1 and 2). This plasmid contains the TMV-U1 126-, 183-, and 30-kDa ORFs, the ToMV coat protein gene (ToMVcp), the SP6 promoter, the rice α-amylase cDNA pOS103, and part of the pBR322 plasmid. The TAA stop codon in the 30-kDa ORF is underlined. The TMV-U1 subgenomic promoter located within the minus strand of the 30-kDa ORF controls the expression of α-amylase. The putative transcription start point (tsp) of the subgenomic RNA is indicated with a period (.).

FIG. 2. Nucleotide sequences of (a) TT01A 103L (SEQ ID NOs: 3 and 4) and (b) the 5′ untranslated leader in TTO1A 103 (SEQ ID NOs: 5 and 6).

FIG. 3. GFP expression vector, TTOSA1 APE pBAD #5. This plasmid contains the TMV-U1 126-, 183-, and 30-kDa ORFs, the ToMV coat protein gene (ToMVcp), the SP6 promoter, the rice α-amylase cDNA pOS103 5′ untranslated leader, GFP, and part of the pBR322 plasmid. The TAA stop codon in the 30-kDa ORF is underlined. The TMV-U1 subgenomic promoter located within the minus strand of the 30-kDa ORF controls the expression of α-amylase. The putative transcription start point (tsp) of the subgenomic RNA is indicated with a period (.).

FIG. 4. Nucleotide sequence of TTOSA1 APE (SEQ ID NOs: 7 and 8).

FIG. 5. 38C13 single chain antibody expression vector, NHL RV. This plasmid contains the TMV-U1 126-, 183-, and 30-kDa ORFs, the ToMV coat protein gene (ToMVcp), the SP6 promoter, the rice α-amylase cDNA pOS103 5′ untranslated leader and signal peptide ORF, murine 38C13 ScFv, and part of the pBR322 plasmid. The TAA stop codon in the 30-kDa ORF is underlined. The TMV-U1 subgenomic promoter located within the minus strand of the 30-kDa ORF controls the expression of α-amylase. The putative transcription start point (tsp) of the subgenomic RNA is indicated with a period (.).

FIG. 6. Nucleotide sequence of BA46 expression vector TTUDABP (SEQ ID NOs: 9 and 10).

FIG. 7. Nucleotide sequence of the hemoglobin expression vector RED1 (SEQ ID NOs: 11 and 12).

### DETAILED DESCRIPTION OF THE INVENTION

The present invention describes the use of non-native 5′ untranslated sequences to enhance RNA or protein production in bacterial, plant or animal hosts. The non-native enhancer sequences may derive from viruses from same or different taxonomic groups. They may also contain sequences from non-viral sources, such as from bacteria, fungi, plants, animals, or other sources. The non-native 5′ untranslated sequences typically have less than about 90%, e.g. less than about 80%, less than about 70%, less than about 60%, less than about 50%, less than about 40%, less than about 30%, less than about 20%, or less than about 10% of sequence homology relative to the native viral sequences. In some embodiments of the instant invention, the 5′ non-native untranslated sequence is a new sequence from a different taxonomic viral group, a non-viral source, a random, or a semi-random sequence inserted into any nucleotide position before the initiation codon of the viral genome.

The non-native 5′ untranslated sequences also encompass analogs of naturally occurring nucleotides. Such analogs include, but are not limited to, phosphoramidates, peptide-nucleic acids, phosphorothioates, methylphosphonates, and the like. In addition to having non-naturally occurring backbones, analogs of naturally occurring polynucleotides may comprise nucleic base analogs, e.g., 7-deazaguanosine, 5-methyl cytosine, inosine, and the like. Descriptions of these analogs and their synthesis can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; 5,262,530; and 5,700,642.

In some embodiments of the invention, the non-native enhancer sequences may be generated by in vitro mutagenesis, recombination or a combination thereof. In vitro methods, including, but not limited to, chemical treatment, oligonucleotide mediated mutagenesis, error-prone PCR, combinatorial cassette mutagenesis, DNA shuffling, random-priming recombination, restriction enzyme fragment induced template switching, staggered extension process, among others. In some embodiments of the instant invention, a library containing sequence variants of the enhancer sequences may be expressed in plant hosts to select the enhancer sequences that confer optimal level of RNA or protein production. A more detailed discussion of methods for generating libraries of nucleic acid sequence variants and selecting desired RNA or protein production level is presented in two co-pending and co-owned U.S. patent application Ser. Nos. 09/359,300 and 09/359,304 both incorporated herein by reference.

The non-native 5′ untranslated sequences or the inserted non-native sequences may be of various lengths. Preferably, the size of non-native nucleic acid sequence or the inserted non-native sequences is from about 5 to 1,000 base pairs (bp), e.g. from about 5 to 500, from about 5 to 200, from about 5 to 100, or from about 10 to 100.

I. Recombinant Plant Viral Nucleic Acids

The construction of viral vectors may use a variety of methods known in the art. In preferred embodiments of the instant invention, the viral vectors are derived from the RNA plant viruses. A variety of plant virus families may be used, such as Bromoviridae, Bunyaviridae, Comoviridae, Geminiviridae, Potyviridae, and Tombusviridae, among others. Within the plant virus families, various genera of viruses may be suitable for the instant invention, such as alfamovirus, ilarvirus, bromovirus, cucumovirus, tospovirus, carlavirus, caulimovirus, closterovirus, comovirus, nepovirus, dianthovirus, furovirus, hordeivirus, luteovirus, necrovirus, potexvirus, potyvirus, rymovirus, bymovirus, oryzavirus, sobemovirus, tobamovirus, tobravirus, carmovirus, tombusvirus, tymovirus, umbravirusa, and among others.

Within the genera of plant viruses, many species are particular preferred. They include alfalfa mosaic virus, tobacco streak virus, brome mosaic virus, broad bean mottle virus, cowpea chlorotic mottle virus, cucumber mosaic virus, tomato spotted wilt virus, carnation latent virus, caulflower mosaic virus, beet yellows virus, cowpea mosaic virus, tobacco ringspot virus, carnation ringspot virus, soil-borne wheat mosaic virus, tomato golden mosaic virus, cassava latent virus, barley stripe mosaic virus, barley yellow dwarf virus, tobacco necrosis virus, tobacco etch virus, potato virus X, potato virus Y, rice necrosis virus, ryegrass mosaic virus, barley yellow mosaic virus, rice ragged stunt virus, Southern bean mosaic virus, tobacco mosaic virus, ribgrass mosaic virus, cucumber green mottle mosaic virus watermelon strain, oat mosaic virus, tobacco rattle virus, carnation mottle virus, tomato bushy stunt virus, turnip yellow mosaic virus, carrot mottle virus, among others. In addition, RNA satellite viruses, such as tobacco necrosis satellite may also be employed.

A given plant virus may contain either DNA or RNA, which may be either single- or double-stranded. One example of plant viruses containing double-stranded DNA includes, but not limited to, caulimoviruses such as cauliflower mosaic virus (CaMV). Representative plant viruses which contain single-stranded DNA are cassava latent virus, bean golden mosaic virus (BGMV), and chloris striate mosaic virus. Rice dwarf virus and wound tumor virus are examples of double-stranded RNA plant viruses. Single-stranded RNA plant viruses include tobacco mosaic virus (TMV), turnip yellow mosaic virus (TYMV), rice necrosis virus (RNV) and brome mosaic virus (BMV). The single-stranded RNA viruses can be further divided into plus sense (or positive-stranded), minus sense (or negative-stranded), or ambisense viruses. The genomic RNA of a plus sense RNA virus is messenger sense, which makes the naked RNA infectious. Many plant viruses belong to the family of plus sense RNA viruses. They include, for example, TMV, BMV, and others. RNA plant viruses typically encode several common proteins, such as replicase/polymerase proteins essential for viral replication and mRNA synthesis, coat proteins providing protective shells for the extracellular passage, and other proteins required for the cell-to-cell movement, systemic infection and self-assembly of viruses. For general information concerning plant viruses, see Matthews, Plant Virology, 3^{rd} Ed., Academic Press, San Diego (1991).

Selected groups of suitable plant viruses are characterized below. However, the invention should not be construed as limited to using these particular viruses, but rather the method of the present invention is contemplated to include all plant viruses at a minimum.

### Tobamovirus Group

Tobacco Mosaic virus (TMV) is a member of the tobamoviruses. The TMV virion is a tubular filament, and comprises coat protein sub-units arranged in a single right-handed helix with the single-stranded RNA intercalated between the turns of the helix. TMV infects tobacco as well as other plants. TMV is transmitted mechanically and may remain infective for a year or more in soil or dried leaf tissue.

The TMV virions may be inactivated by subjection to an environment with a pH of less than 3 or greater than 8, or by formaldehyde or iodine. Preparations of TMV may be obtained from plant tissues by (NH₄)₂SO₄ precipitation, followed by differential centrifugation.

Tobacco mosaic virus (TMV) is a positive-stranded ssRNA virus whose genome is 6395 nucleotides long and is capped at the 5′-end but not polyadenylated. The genomic RNA can serve as mRNA for protein of a molecular weight of about 130,000 (130K) and another produced by read-through of molecular weight about 180,000 (180K). However, it cannot function as a messenger for the synthesis of coat protein. Other genes are expressed during infection by the formation of monocistronic, 3′-coterminal subgenomic mRNAs, including one (LMC) encoding the 17.5K coat protein and another (12) encoding a 30K protein. The 30K protein has been detected in infected protoplasts as described in Miller, J., Virology 132:71 (1984), and it is involved in the cell-to-cell transport of the virus in an infected plant as described by Deom et al., Science 237:389 (1987). The functions of the two large proteins are unknown, however, they are thought to function in RNA replication and transcription.

Several double-stranded RNA molecules, including double-stranded RNAs corresponding to the genomic, 12 and LMC RNAs, have been detected in plant tissues infected with TMV. These RNA molecules are presumably intermediates in genome replication and/or mRNA synthesis processes which appear to occur by different mechanisms.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′- to 5′-direction from the nucleation site).

Another member of the Tobamoviruses, the Cucumber Green Mottle Mosaic virus watermelon strain (CGMMV-W) is related to the cucumber virus (Nozu et al., Virology 45:577 (1971)). The coat protein of CGMMV-W interacts with RNA of both TMV and CGMMV to assemble viral particles in vitro (Kurisu et al., Virology 70:214 (1976)).

Several strains of the tobamovirus group are divided into two subgroups, on the basis of the location of the origin of assembly. Subgroup I, which includes the vulgare, OM, and tomato strain, has an origin of assembly about 800-1000 nucleotides from the 3′-end of the RNA genome, and outside the coat protein cistron (Lebeurier et al., Proc. Natl. Acad. Sci. USA 74:149 (1977); and Fukuda et al., Virology 101 :493 (1980)). Subgroup II, which includes CGMMV-W and cowpea strain (Cc) has an origin of assembly about 300-500 nucleotides from the 3′-end of the RNA genome and within the coat protein cistron. The coat protein cistron of CGMMV-W is located at nucleotides 176-661 from the 3′-end. The 3′ noncoding region is 175 nucleotides long. The origin of assembly is positioned within the coat protein cistron (Meshi et al., Virology 127:54 (1983)).

### Brome Mosaic Virus Group

Brome Mosaic virus (BMV) is a member of a group of tripartite, single-stranded, RNA-containing plant viruses commonly referred to as the bromoviruses. Each member of the bromoviruses infects a narrow range of plants. Mechanical transmission of bromoviruses occurs readily, and some members are transmitted by beetles. In addition to BMV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, single-stranded RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by Ahlquist et al., J. Mol. Biol. 153:23 (1981).

### Rice Necrosis Virus

Rice Necrosis virus is a member of the Potato Virus Y Group or Potyviruses. The Rice Necrosis virion is a flexuous filament comprising one type of coat protein (molecular weight about 32,000 to about 36,000) and one molecule of linear positive-sense single-stranded RNA. The Rice Necrosis virus is transmitted by Polymyxa oraminis (a eukaryotic intracellular parasite found in plants, algae and fungi).

### Geminiviruses

Geminiviruses are a group of small, single-stranded DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, single-stranded DNA. In some geminiviruses (i.e., Cassava latent virus and bean golden mosaic virus) the genome appears to be bipartite, containing two single-stranded DNA molecules.

### Potyviruses

Potyviruses are a group of plant viruses which produce polyprotein. A particularly preferred potyvirus is tobacco etch virus (TEV). TEV is a well characterized potyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

The selection of the genetic backbone for the viral vectors of the instant invention may depend on the plant host used. The plant host may be a monocotyledonous or dicotyledonous plant, plant tissue, or plant cell. Typically, plants of commercial interest, such as food crops, seed crops, oil crops, ornamental crops and forestry crops are preferred. For example, wheat, rice, corn, potato, barley, tobacco, soybean canola, maize, oilseed rape, lilies, grasses, orchids, irises, onions, palms, tomato, the legumes, or Arabidopsis, can be used as a plant host. Host plants may also include those readily infected by an infectious virus, such as Nicotiana, preferably, Nicotiana benthamiana, or Nicotiana clevelandii.

One feature of the present invention is the use of plant viral nucleic acids which comprise one or more non-native nucleic acid sequences capable of being transcribed in a plant host. These nucleic acid sequences may be native nucleic acid sequences that occur in a host plant. Preferably, these nucleic acid sequences are non-native nucleic acid sequences that do not normally occur in a host plant. For example, the plant viral vectors may contain sequences from more than one virus, including viruses from more than one taxonomic group. The plant viral nucleic acids may also contain sequences from non-viral sources, such as foreign genes, regulatory sequences, fragments thereof from bacteria, fungi, plants, animals or other sources. These foreign sequences may encode commercially useful proteins, polypeptides, or fusion products thereof, such as enzymes, antibodies, hormones, pharmaceuticals, vaccines, pigments, anti-microbial peptides and the like. Or they may be sequences that regulate the transcription or translation of viral nucleic acids, package viral nucleic acid, and facilitate systemic infection in the host, among others.

Examples of enzymes that may be produced using the instant invention include, but are not limited to, glucanase, chymosin, proteases, polymerases, saccharidases, deyhdrogenases, nucleases, glucose oxidase, α-amylase, oxidoreductases (such as fungal peroxidases and laccases), xylanases, phytases, cellulases, hemicellulases, and lipases. This invention may also be used to produce enzymes such as, those used in detergents, rennin, horseradish peroxidase, amylases from other plants, soil remediation enzymes, and other such industrial proteins.

Examples of proteins that may be produced using the instant invention include, but are not limited to, blood proteins (e.g., serum albumin, Factor VII, Factor VIII (or modified Factor VIII), Factor IX, Factor X, tissue plasminogen factor, tissue plasminogen activator (t-PA), Protein C, von Willebrand factor, antithrombin III, and erythropoietin (EPO), urokinase, prourokinase, epoetin-α, colony stimulating factors (such as granulocyte colony-stimulating factor (G-CSF), macrophage colony-stimulating factor (M-CSF), and granulocyte macrophage colony-stimulating factor (GM-CSF)), cytokines (such as interleukins or interferons), integrins, addressins, selecting, homing receptors, surface membrane proteins (such as, surface membrane protein receptors), T cell receptor units, immunoglobulins, soluble major histocompatibility complex antigens, structural proteins (such as collagen, fibrin, elastin, tubulin, actin, and myosin), growth factor receptors, growth factors, growth hormone, cell cycle proteins, vaccines, fibrinogen, thrombin, cytokines, hyaluronic acid and antibodies.

In some embodiments of the instant invention, the plant viral vectors may comprise one or more additional native or non-native subgenomic promoters which are capable of transcribing or expressing adjacent nucleic acid sequences in the plant host. These non-native subgenomic promoters are inserted into the plant viral nucleic acids without destroying the biological function of the plant viral nucleic acids using known methods in the art. For example, the CaMV promoter can be used when plant cells are to be transfected. The subgenomic promoters are capable of functioning in the specific host plant. For example, if the host is tobacco, TMV, tomato mosaic virus, or other viruses containing subgenomic promoter may be utilized. The inserted subgenomic promoters should be compatible with the TMV nucleic acid and capable of directing transcription or expression of adjacent nucleic acid sequences in tobacco. It is specifically contemplated that two or more heterologous non-native subgenomic promoters may be used. The non-native nucleic acid sequences may be transcribed or expressed in the host plant under the control of the subgenomic promoter to produce the products of the nucleic acids of interest.

In some embodiments of the instant invention, the recombinant plant viral nucleic acids may be further modified by conventional techniques to delete all or part of the native coat protein coding sequence or put the native coat protein coding sequence under the control of a non-native plant viral subgenomic promoter. If it is deleted or otherwise inactivated, a non-native coat protein coding sequence is inserted under control of one of the non-native subgenomic promoters, or optionally under control of the native coat protein gene subgenomic promoter. Thus, the recombinant plant viral nucleic acid contains a coat protein coding sequence, which may be native or a nonnative coat protein coding sequence, under control of one of the native or non-native subgenomic promoters. The native or non-native coat protein gene may be utilized in the recombinant plant viral nucleic acid. The non-native coat protein, as is the case for the native coat protein, may be capable of encapsidating the recombinant plant viral nucleic acid and providing for systemic spread of the recombinant plant viral nucleic acid in the host plant.

In some embodiments of the instant invention, recombinant plant viral vectors are constructed to express a fusion between a plant viral coat protein and the foreign genes or polypeptides of interest. Such a recombinant plant virus provides for high level expression of a nucleic acid of interest. The location(s) where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint which sometimes does not result in translational termination.

In some embodiments of the instant invention, nucleic sequences encoding reporter protein(s) or antibiotic/herbicide resistance gene(s) may be constructed as carrier protein(s) for the polypeptides of interest, which may facilitate the detection of polypeptides of interest. For example, green fluorescent protein (GFP) may be simultaneously expressed with polypeptides of interest. In another example, a reporter gene, β-glucuronidase (GUS) may be utilized. In another example, a drug resistance marker, such as a gene whose expression results in kanamycin resistance, may be used.

Since the RNA genome is typically the infective agent, the cDNA is positioned adjacent a suitable promoter so that the RNA is produced in the production cell. The RNA is capped using conventional techniques, if the capped RNA is the infective agent. In addition, the capped RNA can be packaged in vitro with added coat protein from TMV to make assembled virions. These assembled virions can then be used to inoculate plants or plant tissues. Alternatively, an uncapped RNA may also be employed in the embodiments of the present invention. Contrary to the practiced art in scientific literature and in issued patent (Ahlquist et al., U.S. Pat. No. 5,466,788), uncapped transcripts for virus expression vectors are infective on both plants and in plant cells. Capping is not a prerequisite for establishing an infection of a virus expression vector in plants, although capping increases the efficiency of infection. In addition, nucleotides may be added between the transcription start site of the promoter and the start of the cDNA of a viral nucleic acid to construct an infectious viral vector. One or more nucleotides may be added. In some embodiments of the present invention, the inserted nucleotide sequence may contain a G at the 5′-end. Alternatively, the inserted nucleotide sequence may be GNN, GTN, or their multiples, (GNN)ₓ or (GTN)ₓ.

In some embodiments of the instant invention, more than one nucleic acid is prepared for a multipartite viral vector construct. In this case, each nucleic acid may require its own origin of assembly. Each nucleic acid could be prepared to contain a subgenomic promoter and a non-native nucleic acid. Alternatively, the insertion of a non-native nucleic acid into the nucleic acid of a monopartite virus may result in the creation of two nucleic acids (i.e., the nucleic acid necessary for the creation of a bipartite viral vector). This would be advantageous when it is desirable to keep the replication and transcription or expression of the nucleic acid of interest separate from the replication and translation of some of the coding sequences of the native nucleic acid.

The recombinant plant viral nucleic acid may be prepared by cloning a viral nucleic acid. If the viral nucleic acid is DNA, it can be cloned directly into a suitable vector using conventional techniques. One technique is to attach an origin of replication to the viral DNA which is compatible with the cell to be transfected. In this manner, DNA copies of the chimeric nucleotide sequence are produced in the transfected cell. If the viral nucleic acid is RNA, a DNA copy of the viral nucleic acid is first prepared by well-known procedures. For example, the viral RNA is transcribed into DNA using reverse transcriptase to produce subgenomic DNA pieces, and a double-stranded DNA may be produced using DNA polymerases. The cDNA is then cloned into appropriate vectors and cloned into a cell to be transfected. In some instances, cDNA is first attached to a promoter which is compatible with the production cell. The recombinant plant viral nucleic acid can then be cloned into any suitable vector which is compatible with the production cell. Alternatively, the recombinant plant viral nucleic acid is inserted in a vector adjacent a promoter which is compatible with the production cell. In some embodiments, the cDNA ligated vector may be directly transcribed into infectious RNA in vitro and inoculated onto the plant host. The cDNA pieces are mapped and combined in proper sequence to produce a full-length DNA copy of the viral RNA genome, if necessary.

In some embodiments of the instant invention, increased representation of gene sequences in virus expression libraries may be achieved by bypassing the genetic bottleneck of propagation in bacterial cells. For example, in some embodiments of the instant invention, cell-free methods may be used to assemble sequence libraries or individual arrayed sequences into virus expression vectors and reconstruct an infectious virus, such that the final ligation product can be transcribed and the resulting RNA can be used for plant, plant tissue or plant cell inoculation/infection. A more detailed discussion is presented in a co-pending and co-owned U.S. patent application Ser. No. 09/359,303 incorporated herein by reference.

Those skilled in the art will understand that these embodiments are representative only of many constructs suitable for housing libraries of sequence variants. All such constructs are contemplated and intended to be within the scope of the present invention. The invention is not intended to be limited to any particular viral constructs but specifically contemplates using all operable constructs. A person skilled in the art will be able to construct the plant viral nucleic acids based on molecular biology techniques well known in the art. Suitable techniques have been described in Sambrook et al. (2nd ed.), Cold Spring Harbor Laboratory, Cold Spring Harbor (1989); Methods in Enzymol. (Vols. 68, 100, 101, 118, and 152-155) (1979, 1983, 1986 and 1987); and DNA Cloning, D. M. Clover, Ed., IRL Press, Oxford (1985); Walkey, Applied Plant Virology, Chapman & Hall (1991); Matthews, Plant Virology, 3³ Ed., Academic Press, San Diego (1991); Turpen et al., J. of Virological Methods, 42:227-240 (1993); U.S. Pat. Nos. 4,885,248, 5,173,410, 5,316,931, 5,466,788, 5,491,076, 5,500,360, 5,589,367, 5,602,242, 5,627,060, 5,811,653, 5,866,785, 5,889,190, and 5,589,367, U.S. patent application Ser. No. 08/324,003. Nucleic acid manipulations and enzyme treatments are carried out in accordance with manufacturers' recommended procedures in making such constructs.

Viral nucleic acids containing non-native 5′ untranslated sequence or artificial leader sequence can be transfected as populations or individual clones into host: 1) protoplasts; 2) whole plants; or 3) plant tissues, such as leaves of plants (Dijkstra et al., Practical Plant Virology: Protocols and Exercises, Springer Verlag (1998); Plant Virology Protocol: From Virus Isolation to Transgenic Resistance in Methods in Molecular Biology, Vol. 81, Foster and Taylor, Ed., Humana Press (1998)). The plant host may be a monocotyledonous or dicotyledonous plant, plant tissue, or plant cell. Typically, plants of commercial interest, such as food crops, seed crops, oil crops, ornamental crops and forestry crops are preferred. For example, wheat, rice, corn, potato, barley, tobacco, soybean canola, maize, oilseed rape, lilies, grasses, orchids, irises, onions, palms, tomato, the legumes, or Arabidopsis, can be used as a plant host. Host plants may also include those readily infected by an infectious virus, such as Nicotiana, preferably, Nicotiana benthamiana, or Nicotiana clevelandii.

In some embodiments of the instant invention, the delivery of the plant virus expression vectors into the plant may be affected by the inoculation of in vitro transcribed RNA, inoculation of virions, or internal inoculation of plant cells from nuclear cDNA, or the systemic infection resulting from any of these procedures. In all cases, the co-infection may lead to a rapid and pervasive systemic expression of the desired nucleic acid sequences in plant cells. The systemic infection of the plant by the foreign sequences may be followed by the growth of the infected host to produce the desired product, and the isolation and purification of the desired product, if necessary. The growth of the infected host is in accordance with conventional techniques, as is the isolation and the purification of the resultant products.

The host can be infected with a recombinant viral nucleic acid or a recombinant plant virus by conventional techniques. Suitable techniques include, but are not limited to, leaf abrasion, abrasion in solution, high velocity water spray, and other injury of a host as well as imbibing host seeds with water containing the recombinant viral RNA or recombinant plant virus. More specifically, suitable techniques include:

(a) Hand Inoculations. Hand inoculations are performed using a neutral pH, low molarity phosphate buffer, with the addition of celite or carborundum (usually about 1%). One to four drops of the preparation is put onto the upper surface of a leaf and gently rubbed.

(b) Mechanized Inoculations of Plant Beds. Plant bed inoculations are performed by spraying (gas-propelled) the vector solution into a tractor-driven mower while cutting the leaves. Alternatively, the plant bed is mowed and the vector solution sprayed immediately onto the cut leaves.

(c) High Pressure Spray of Single Leaves. Single plant inoculations can also be performed by spraying the leaves with a narrow, directed spray (50 psi, 6-12 inches from the leaf) containing approximately 1% carborundum in the buffered vector solution.

(d) Vacuum Infiltration. Inoculations may be accomplished by subjecting a host organism to a substantially vacuum pressure environment in order to facilitate infection.

(e) High Speed Robotics Inoculation. Especially applicable when the organism is a plant, individual organisms may be grown in mass array such as in microtiter plates. Machinery such as robotics may then be used to transfer the nucleic acid of interest.

(f) Ballistics (High Pressure Gun) Inoculation. Single plant inoculations can also be performed by particle bombardment. A ballistics particle delivery system (BioRad Laboratories, Hercules, (A) can be used to transfect plants such as N. benthamiana as described previously (Nagar et al., Plant Cell, 7:705-719 (1995)).

An alternative method for introducing viral nucleic acids into a plant host is a technique known as agroinfection or Agrobacterium-mediated transformation (also known as Agro-infection) as described by Grimsley et al., Nature 325:177 (1987). This technique makes use of a common feature of Agrobacterium which colonizes plants by transferring a portion of their DNA (the T-DNA) into a host cell, where it becomes integrated into nuclear DNA. The T-DNA is defined by border sequences which are 25 base pairs long, and any DNA between these border sequences is transferred to the plant cells as well. The insertion of a recombinant plant viral nucleic acid between the T-DNA border sequences results in transfer of the recombinant plant viral nucleic acid to the plant cells, where the recombinant plant viral nucleic acid is replicated, and then spreads systemically through the plant. Agro-infection has been accomplished with potato spindle tuber viroid (PSTV) (Gardner et al., Plant Mol. Biol. 6:221 (1986); CaV (Grimsley et al., Proc. Natl. Acad. Sci. USA 83:3282 (1986)); MSV (Grimsley et al., Nature 325:177 (1987)), and Lazarowitz, S., Nucl. Acids Res. 16:229 (1988)) digitaria streak virus (Donson et al., Virology 162:248 (1988)), wheat dwarf virus (Hayes et al., J. Gen. Virol. 69:891 (1988)) and tomato golden mosaic virus (TGMV) (Elner et al., Plant Mol. Biol. 10:225 (1988) and Gardiner et al., EMBO J. 7:899 (1988)). Therefore, agro-infection of a susceptible plant could be accomplished with a virion containing a recombinant plant viral nucleic acid based on the nucleotide sequence of any of the above viruses. Particle bombardment or electrosporation or any other methods known in the art may also be used.

In some embodiments of the instant invention, infection may also be attained by placing a selected nucleic acid sequence into an organism such as E. coli, or yeast, either integrated into the genome of such organism or not, and then applying the organism to the surface of the host organism. Such a mechanism may thereby produce secondary transfer of the selected nucleic acid sequence into a host organism. This is a particularly practical embodiment when the host organism is a plant. Likewise, infection may be attained by first packaging a selected nucleic acid sequence in a pseudovirus. Such a method is described in WO 94/10329. Though the teachings of this reference may be specific for bacteria, those of skill in the art will readily appreciate that the same procedures could easily be adapted to other organisms.

II. Recombinant Bacterial or Animal Viral Nucleic Acids

One skilled in the art will appreciate that the viral nucleic acids may also be derived from a variety of bacterial or animal viruses, such as M13, ØX174, MS2, T4, lamda, T7, Mu, alphavirus, rhinovirus, poliovirus, polyomavirus, simian virus 40, and adenovirus, among others. Selected groups of bacterial viruses are discussed in Brock et al., Biology of Microorganisms, pp. 263-284, Prentice-Hall Inc., Upper Saddle River, N.J. (1997). Selected groups of suitable viruses are characterized below and in a co-pending and co-owned U.S. patent application Ser. No. ______ (Kumagai et al., Attorney Docket No. 08010137US10, filed herewith, incorporated herein by reference). However, the invention should not be construed as limited to using these particular viruses, but rather the method of the present invention is contemplated to include all animal viruses at a minimum. Recombinant viral nucleic acids comprising non-native 5′untranslated sequences may be obtained using conventional molecular biology techniques (Sambrook et al. (2nd ed.), Cold Spring Harbor Laboratory, Cold Spring Harbor (1989)). Methods for producing recombinant protein or polypeptide in bacterial or animal hosts are also known to those skilled in the art (Sambrook et al. (2nd ed.), Cold Spring Harbor Laboratory, Cold Spring Harbor (1989)).

### Alphaviruses

The alphaviruses are a genus of the viruses of the family Togaviridae. Almost all of the members of this genus are transmitted by mosquitoes, and may cause diseases in man or animals. Some of the alphaviruses are grouped into three serologicallly defined complexes. The complex-specific antigen is associated with the E1 protein of the virus, and the species-specific antigen is associated with the E2 protein of the virus.

The Semliki Forest virus complex includes Bebaru virus, Chikungunya Fever virus, Getah virus, Mayaro Fever virus, O'nyongnyong Fever virus, Ross River virus, Sagiyama virus, Semliki Forest virus and Una virus. The Venezuelan Equine Encephalomyelitis virus complex includes Cabassou virus, Everglades virus, Mucambo virus, Pixuna virus and Venezuelan Equine Encephalomyelitis virus. The Western Equine Encephalomyelitis virus complex includes Aura virus, Fort Morgan virus, Highlands J virus, Kyzylagach virus, Sindbis virus, Western Equine Encephalomyelitis virus and Whataroa virus.

The alphaviruses contain an icosahedral nucleocapsid consisting of 180 copies of a single species of capsid protein complexed with a plus-stranded mRNA. The alphaviruses mature when preassembled nucleocapsid is surrounded by a lipid envelope containing two virus-encoded integral membrane glycoproteins, called E1 and E2. The envelope is acquired when the capsid, assembled in the cytoplasm, buds through the plasma membrane. The envelope consists of a lipid bilayer derived from the host cell.

The mRNA encodes a glycoprotein which is cotranslationally cleaved into nonstructural proteins and structural proteins. The 3′ one-third of the RNA genome consists of a 26S mRNA which encodes for the capsid protein and the E3, E2, K6 and E1 glycoproteins. The capsid is cotranslationally cleaved from the E3 protein. It is hypothesized that the amino acid triad of His, Asp and Ser at the COOH terminus of the capsid protein comprises a serine protease responsible for cleavage. Hahn et al., Proc. Natl. Acad. Sci. USA 82:4648 (1985). Cotranslational cleavage also occurs between E2 and K proteins. Thus, two proteins PE2 which consists of E3 and E2 prior to cleavage and an El protein comprising K6 and E1 are formed. These proteins are cotranslationally inserted into the endoplasmic reticulum of the host cell, glycosylated and transported via the Golgi apparatus to the plasma membrane where they can be used for budding. At the point of virion maturation the E3 and E2 proteins are separated. The E1 and E2 proteins are incorporated into the lipid envelope.

It has been suggested that the basic amino-terminal half of the capsid protein stabilizes the interaction of capsid with genomic RNA or interacts with genomic RNA to initiate a encapsidation, Strauss et al., in the Togaviridae and Flaviviridaei, Ed. S. Schlesinger & M. Schlesinger, Plenum Press, New York, pp. 35-90 (1980). These suggestions imply that the origin of assembly is located either on the unencapsidated genomic RNA or at the amino-terminus of the capsid protein. It has been suggested that E3 and K6 function as signal sequences for the insertion of PE2 and E1, respectively, into the endoplasmic reticulum.

Work with temperature sensitive mutants of alphaviruses has shown that failure of cleavage of the structural proteins results in failure to form mature virions. Lindquist et al., Virology 151:10 (1986) characterized a temperature sensitive mutant of Sindbis virus, t_{S} 20. Temperature sensitivity results from an A-U change at nucleotide 9502. The t_{S} lesion present cleavage of PE2 to E2 and E3 and the final maturation of progeny virions at the nonpermissive temperature. Hahn et al., supra, reported three temperature sensitive mutations in the capsid protein which prevents cleavage of the precursor polyprotein at the nonpermissive temperature. The failure of cleavage resulted in no capsid formation and very little envelope protein.

Defective interfering RNAs (DI particles) of Sindbis virus are helper-dependent deletion mutants which interfere specifically with the replication of the homologous standard virus. Perrault, J., Microbiol. Immunol. 93:151 (1981). DI particles have been found to be functional vectors for introducing at least one foreign gene into cells. Levis, R., Proc. Natl. Acad. Sci. USA 84:4811 (1987).

It has been found that it is possible to replace at least 1689 internal nucleotides of a DI genome with a foreign sequence and obtain RNA that will replicate and be encapsidated. Deletions of the DI genome do not destroy biological activity. The disadvantages of the system are that DI particles undergo apparently random rearrangements of the internal RNA sequence and size alterations. Monroe et al., J. Virology 49:865 (1984). Expression of a gene inserted into the internal sequence is not as high as expected. Levis et al., supra, found that replication of the inserted gene was excellent but translation was low. This could be the result of competition with whole virus particles for translation sites and/or also from disruption of the gene due to rearrangement through several passages.

Two species of mRNA are present in alphavirus-infected cells: A 42S mRNA region, which is packaged into nature virions and functions as the message for the nonstructural proteins, and a 26S mRNA, which encodes the structural polypeptides. the 26S mRNA is homologous to the 3′ third of the 42S mRNA. It is translated into a 130K polyprotein that is cotranslationally cleaved and processed into the capsid protein and two glycosylated membrane proteins, E1 and E2.

The 26S mRNA of Eastern Equine Encephalomyelitis (EEE) strain 82V-2137 was cloned and analyzed by Chang et al., J. Gen. Virol. 68:2129 (1987). The 26S mRNA region encodes the capsid proteins, E3, E2, 6K and E1. The amino terminal end of the capsid protein is thought to either stabilize the interaction of capsid with mRNA or to interact with genomic RNA to initiate encapsidation.

Uncleaved E3 and E2 proteins called PE2 is inserted into the host endoplasmic reticulum during protein synthesis. The PE2 is thought to have a region common to at least five alphaviruses which interacts with the viral nucleocapsid during morphogenesis.

The 6K protein is thought to function as a signal sequence involved in translocation of the E1 protein through the membrane. The E1 protein is thought to mediate virus fusion and anchoring of the E1 protein to the virus envelope.

### Rhinoviruses

The rhinoviruses are a genus of viruses of the family Picornaviridae. The rhinoviruses are acid-labile, and are therefore rapidly inactivated at pH values of less than about 6. The rhinoviruses commonly infect the upper respiratory tract of mammals.

Human rhinoviruses are the major causal agents of the common cold, and many serotypes are known. Rhinoviruses may be propagated in various human cell cultures, and have an optimum growth temperature of about 33° C. Most strains of rhinoviruses are stable at or below room temperature and can withstand freezing. Rhinoviruses can be inactivated by citric acid, tincture of iodine or phenol/alcohol mixtures.

The complete nucleotide sequence of human rhinovirus 2 (HRV2) has been sequenced. The genome consists of 7102 nucleotides with a long open reading frame of 6450 nucleotides which is initiated 611 nucleotides from the 5′-end and stops 42 nucleotides from the poly(A) tract. Three capsid proteins and their cleavage cites have been identified.

Rhinovirus RNA is single-stranded and positive-sense. The RNA is not capped, but is joined at the 5′-end to a small virus-encoded protein, virion-protein genome-linked (VPg). Translation is presumed to result in a single polyprotein which is broken by proteolytic cleavage to yield individual virus proteins. An icosahedral viral capsid contains 60 copies each of 4 virus proteins VP 1, VP2, VP3 and VP4 and surrounds the RNA genome. Medappa, K., Virology 44:259 (1971).

Analysis of the 610 nucleotides preceding the long open reading frame shows several short open reading frames. However, no function can be assigned to the translated proteins since only two sequences show homology throughout HRV2, HRV14 and the 3 sterotypes of poliovirus. These two sequences may be critical in the life cycle of the virus. They are a stretch of 16 bases beginning at 436 in HRV2 and a stretch of 23 bases beginning at 531 in HRV2. Cutting or removing these sequences from the remainder of the sequence for non-structural proteins could have an unpredictable effect upon efforts to assemble a mature virion.

The capsid proteins of HRV2: VP4, VP2, VP3 and VP1 begin at nucleotide 611, 818, 1601 and 2311, respectively. The cleavage point between VP1 and P2A is thought to be around nucleotide 3255. Skern et al., Nucleic Acids Research 13:2111 (1985).

Human rhinovirus type 89 (HRV89) is very similar to HRV2. It contains a genome of 7152 nucleotides with a single large open reading frame of 2164 condons. Translation begins at nucleotide 619 and ends 42 nucleotides before the poly(A) tract. The capsid structural proteins, VP4, VP2, VP3 and VP1 are the first to be translated. Translation of VP4 begins at 619. Cleavage cites occur at:

| | | |
|---|---|---|
| VP4/VP2 | 825 | determined |
| VP2/VP3 | 1627 | determined |
| VP3/VP1 | 2340 | determined |
| VP1/P2-A | 3235 | presumptive |

Duechler et al., Proc. Natl. Acad. Sci. USA 84:2605 (1987).

### Polioviruses

Polioviruses are the causal agents of poliomyelitis in man, and are one of three groups of enteroviruses. Enteroviruses are a genus of the family Picornaviridae (also the family of rhinoviruses). Most enteroviruses replicate primarily in the mammalian gastrointestinal tract, although other tissues may subsequently become infected. Many enteroviruses can be propagated in primarily cultures of human or monkey kidney cells and in some cell lines (e.g. HeLa, Vero, WI-e8). Inactivation of the enteroviruses may be accomplished with heat (about 50° C.), formaldehyde (3%), hydrochloric acid (01.N) or chlorine (ca. 0.3-0.5 ppm free residual Cl₂).

The complete nucleotide sequence of poliovirus PV2 (Sab) and PV3 (Sab) have been determined. They are 7439 and 7434 nucleotide in length, respectively. There is a single long open reading frame which begins more than 700 nucleotides from the 5′-end. Poliovirus translation produces a single polyprotein which is cleaved by proteolytic processing. Kitamura et al., Nature 291:547 (1981).

It is speculated that these homologous sequences in the untranslated regions play an essential role in viral replication such as:

1. viral-specific RNA synthesis;

2. viral-specific protein synthesis; and

3. packaging

Toyoda, H. et al., J. Mol. Biol. 174:561 (1984).

The structures of the serotypes of poliovirus have a high degree of sequence homology. Their coding sequences code for the same proteins in the same order. Therefore, genes for structural proteins are similarly located. In PV1, PV2 and PV3, the polyprotein begins translation near the 750 nucleotide. The four structural proteins VP4, VP2, VP3 and VP1 begin at about 745, 960, 1790 and 2495, respectively, with VP1 ending at about 3410. They are separated in vivo by proteolytic cleavage, rather than by stop/start codons.

### Simian Virus 40

Simian virus 40 (SV40) is a virus of the genus Polyomavirus, and was originally isolated from the kidney cells of the rhesus monkey. The virus is commonly found, in its latent form, in such cells. Simian virus 40 is usually non-pathogenic in its natural host.

Simian virus 40 virions are made by the assembly of three structural proteins, VP1, VP2 and VP3. Girard et al., Biochem. Biophys. Res. Commun. 40:97 (1970); Prives et al., Proc. Natl. Acad. Sci. USA 71 :302 (1974); and Jacobson et al., Proc. Natl. Acad. Sci. USA 73:2742-2746 (1976). The three corresponding viral genes are organized in a partially overlapping manner. They constitute the late genes portion of the genome. Tooze, J., Molecular Biology of Tumor Viruses Appendix A The SV40 Nucleotide Sequence, 2nd Ed. Part 2, pp. 799-829 (1980), Cold Spring Harbor Laboratory, Cold Spring Harbor, N.Y. Capsid proteins VP2 and VP3 are encoded by nucleotides 545 to 1601 and 899 to 1601, respectively, and both are read in the same frame. VP3 is therefore a subset of VP2. Capsid protein VP1 is encoded by nucleotides 1488-2574. The end of the VP2-VP3 open reading frame therefore overlaps the VP1 by 113 nucleotides but is read in an alternative frame. Tooze, J., supra. Wychowski et al., J. Virology 61:3862 (1987).

### Adenoviruses

Adenovirus type 2 is a member of the adenovirus family or adenovirus. This family of viruses are non-enveloped, icosahedral, linear, double-stranded DNA-containing viruses which infect mammals or birds.

The adenovirus virion consists of an icosahedral capsid enclosing a core in which the DNA genome is closely associated with a basic (arginine-rich) viral polypeptide VII. The capsid is composed of 252 capsomeres: 240 hexons (capsomers each surrounded by 6 other capsomers) and 12 pentons (one at each vertex, each surrounded by 5 ‘peripentonal’ hexons). Each penton consists of a penton base (composed of viral polypeptide III) associated with one (in mammalian adenoviruses) or two (in most avian adenoviruses) glycoprotein fibres (viral polypeptide IV). The fibres can act as haemagglutinins and are the sites of attachment of the virion to a host cell-surface receptor. The hexons each consist of three molecules of viral polypeptide II; they make up the bulk of the icosahedron. Various other minor viral polypeptides occur in the virion.

The adenovirus dsDNA genome is covalently linked at the 5′-end of each strand to a hydrophobic ‘terminal protein’, TP (molecular weight about 55,000 Da); the DNA has an inverted terminal repeat of different length in different adenoviruses. In most adenoviruses examined, the 5′-terminal residue is dCMP.

During its replication cycle, the virion attaches via its fibres to a specific cell-surface receptor, and enters the cell by endocytosis or by direct penetration of the plasma membrane. Most of the capsid proteins are removed in the cytoplasm. The virion core enters the nucleus, where the uncoating is completed to release viral DNA almost free of virion polypeptides. Virus gene expression then begins. The viral dsDNA contains genetic information on both strands. Early genes (regions E1a, E1b, E2a, E3, E4) are expressed before the onset of viral DNA replication. Late genes (regions L1, L2, L3, L4 and L5) are expressed only after the initiation of DNA synthesis. Intermediate genes (regions E2b and Iva₂) are expressed in the presence or absence of DNA synthesis. Region E1a encodes proteins involved in the regulation of expression of other early genes, and is also involved in transformation. The RNA transcripts are capped (with m⁷G⁵ppp⁵N) and polyadenylated in the nucleus before being transferred to the cytoplasm for translation.

Viral DNA replication requires the terminal protein, TP, as well as virus-encoded DNA polymerase and other viral and host proteins. TP is synthesized as an 80K precursor, pTP, which binds covalently to nascent replicating DNA strands. pTP is cleaved to the mature 55K TP late in virion assembly; possibly at this stage, pTP reacts with a dCTP molecule and becomes covalently bound to a dCMP residue, the 3′ OH of which is believed to act as a primer for the initiation of DNA synthesis. Late gene expression, resulting in the synthesis of viral structural proteins, is accompanied by the cessation of cellular protein synthesis, and virus assembly may result in the production of up to 105 virions per cell.

In order to provide a clear and consistent understanding of the specification and the claims, including the scope given herein to such terms, the following definitions are given: 5′ untranslated sequences: sequences at the 5′ end of a viral genome up to the initiation codon.

Coat protein (capsid protein): an outer structural protein of a virus.

Gene: a discrete nucleic acid sequence responsible for a discrete cellular product.

Host: a cell, tissue or organism capable of replicating a vector or viral nucleic acid and which is capable of being infected by a virus containing the viral vector or viral nucleic acid. This term is intended to include prokaryotic and eukaryotic cells, organs, tissues, organisms, or in vitro extracts thereof, where appropriate.

Infection: the ability of a virus to transfer its nucleic acid to a host or introduce viral nucleic acid into a host, wherein the viral nucleic acid is replicated, viral proteins are synthesized, and new viral particles assembled.

Movement protein: a noncapsid protein required for cell-to-cell movement of RNA replicons or viruses in plants.

Non-native (foreign): any sequence that does not normally occur in the virus or its host or does not occur at its normal location in the viral or its host genome.

Open Reading Frame: a nucleotide sequence of suitable length in which there are no stop codons.

Plant Cell: the structural and physiological unit of plants, consisting of a protoplast and the cell wall.

Plant Tissue: any tissue of a plant in planta or in culture. This term is intended to include a whole plant, plant cell, plant organ, protoplast, cell culture, or any group of plant cells organized into a structural and functional unit.

Promoter: the 5′-flanking, non-coding sequence adjacent to a coding sequence which is involved in the initiation of transcription of the coding sequence.

Protoplast: an isolated cell without cell walls, having the potency for regeneration into cell culture or a whole host.

Subgenomic mRNA promoter: a promoter that directs the synthesis of an mRNA smaller than the full-length genome in size.

Vector: a self-replicating nucleic acid molecule that contains non-native sequences and which transfers nucleic acid segments between cells.

Virion: a particle composed of viral nucleic acid, viral coat protein (or capsid protein).

Virus: an infectious agent composed of a nucleic acid encapsulated in a protein.

### EXAMPLES OF THE PREFERRED EMBODIMENTS

The following examples further illustrate the present invention. These examples are intended merely to be illustrative of the present invention and are not to be construed as being limiting. −Example 1

Construction of the Rice Alpha-Amylase Expression Vector TTO1A 103.

Unique XhoI, AvrII sites were inserted into the rice α-amylase OS103 cDNA by polymerase chain reaction (PCR) mutagenesis using oligonucleotides: 5′-GCC TCG AGT GCA CCA TGC AGG TGC TGA ACA CCA TGG TG-3′ (upstream) (SEQ ID NO: 13) and 5′-TCC CTA GGT CAG ATT TTC TCC CAG ATT GCG TAG C-3′ (downstream) (SEQ ID NO: 14). The 1.4-kb XoI, AvrII OS103 PCR fragment was subcloned into pTTO1A, creating plasmid TTO1A 103. Plasmid TTO1A 103 has been deposited in American Type Culture Collection (assigned PTA-333).

Construction of the Rice Alpha-Amylase Expression Vector TTO1A 103L

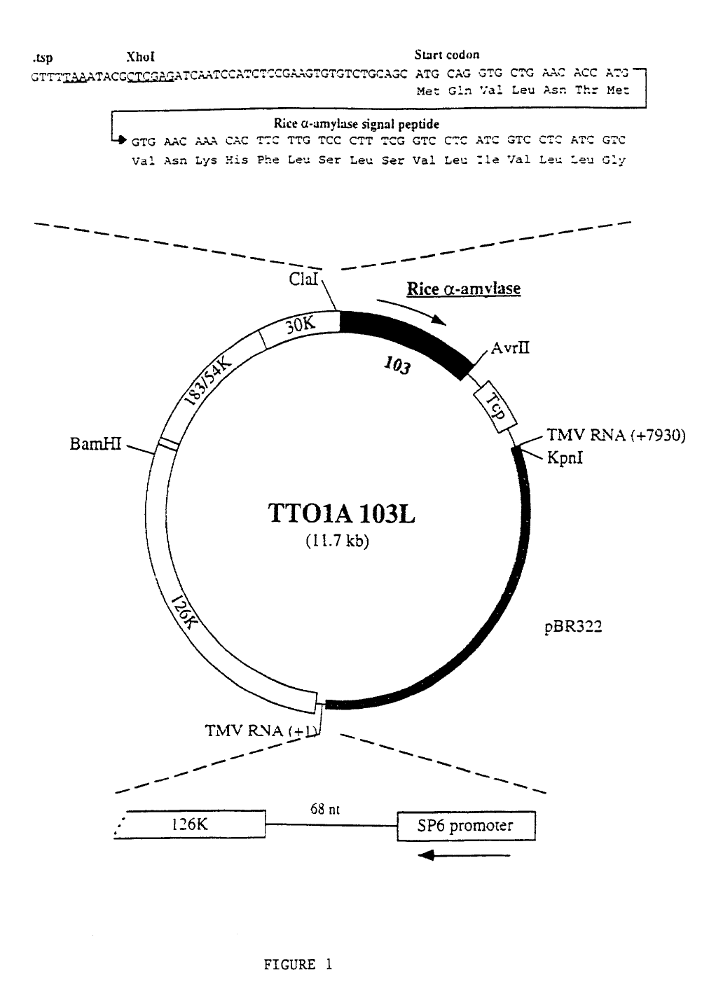

Unique XhoI, AvrII sites were inserted into the rice α-amylase pOS103 cDNA by polymerase chain reaction (PCR) mutagenesis using oligonucleotides: 5′-CTC TCG AGA TCA ATC ATC CAT CTC CGA AGT GTG TCT GC-3′ (upstream) (SEQ ID NO: 15) and 5′-TCC CTA GGT CAG ATT TTC TCC CAG ATT GCG TAG C-3′ (downstream) (SEQ ID NO: 16). The 1.4-kb XhoI, AvrII OS103 PCR fragment was subcloned into pTTO1A (Kumagai et al., Proc. Natl. Acad. Sci. USA 92:1679-1683 (1995)), creating plasmid TTO1A 103L (FIG. 1). Plasmid TTO1A 103L has been deposited in American Type Culture Collection (assigned PTA-327).

In vitro Transcriptions, Inoculations, and Analysis of Transfected Plants

N. benthamiana plants were inoculated with in vitro transcripts of KpnI-digested TTO1A 103, TTO1A 103L as described in Kumagai et al., Proc. Natl. Acad. Sci. USA 92:1679-1683 (1995). Virions were isolated from N. benthamiana leaves infected with TTO1A 103L transcripts and stained with 2% aqueous uranyl acetate. Transmission electron micrographs were taken using a ZeiSS™ CEM902® instrument.

Purification, Immunological Detection, and in vitro Assay of α-Amylase

Ten days after inoculation, total soluble protein was isolated from 10 g of upper, noninoculated N. benthamiana leaf tissue transfected with TTO1A 103L. The leaves were frozen in liquid nitrogen and ground in 20 ml of 10 mM 2-mercaptoethanol/10 mM Tris-bis propane, pH 6.0. The suspension was centrifuged and the supernatant, containing recombinant α-amylase, was bound to a POROS 50 HQ® ion exchange column (PerSeptive Biosystems™). The α-amylase was eluted with a linear gradient of 0-1.0 M NaCl in 50 mM Tris-bis propane pH 7.0. The α-amylase eluted in fraction 16, 17 and its enzyme activity was analyzed (Sigma™ Kit #576-3). Fractions containing cross-reacting material to α-amylase antibody were concentrated with a Centriprep-30® (Amicon™) and the buffer was exchanged by diafiltration (50 mM Tris-bis propane, pH 7.0). The sample was then loaded on a POROS HQ/M® column (Perceptive Biosystems™), eluted with a linear gradient of 0-1.0 M NaCl in 50 mM Tris-bis propane pH 7.0, and assayed for α-amylase activity. Fractions containing cross-reacting material to α-amylase antibody were concentrated with a Centriprep-30® and the buffer was exchanged by diafiltration (20 mM Sodium Acetate/HEPES/MES, pH 6.0). The sample was finally loaded on a POROS HS/M® column (Perceptive Biosystems™), eluted with a linear gradient of 0-1.0 M NaCl in 20 mM Sodium Acetate/HEPES/MES, pH 6.0, and assayed for α-amylase activity. Total soluble plant protein concentrations were determined using bovine serum albumin as a standard. The proteins were analyzed on a 0.1% SDS/10% polyacrylamide gel and transferred by electroblotting for 1 hr to a nitrocellulose membrane. The blotted membrane was incubated for 1 hr with a 2000-fold dilution of anti-α-amylase antiserum. Using standard protocols, the antisera was raised in rabbits against S. cerevisiae expressed rice α-amylase. The enhanced chemiluminescence horseradish peroxidase-linked, goat anti-rabbit IgG assay (Cappel Laboratories™) was performed according to the manufacturer's (Amersham™) specifications. The blotted membrane was subjected to film exposure times of up to 10 sec. The quantity of total recombinant α-amylase in an extracted leaf sample was determined (using a 1-sec exposure of the blotted membrane) by comparing the crude extract chemiluminescent signal to the signal obtained from known quantities of α-amylase. Shorter and longer chemiluminescent exposure times of the blotted membrane gave the same quantitative results.

Comparision of N. benthamiana Transfected with TTO1A 103 and N. benthamiana Transfected with TTO1A 103L

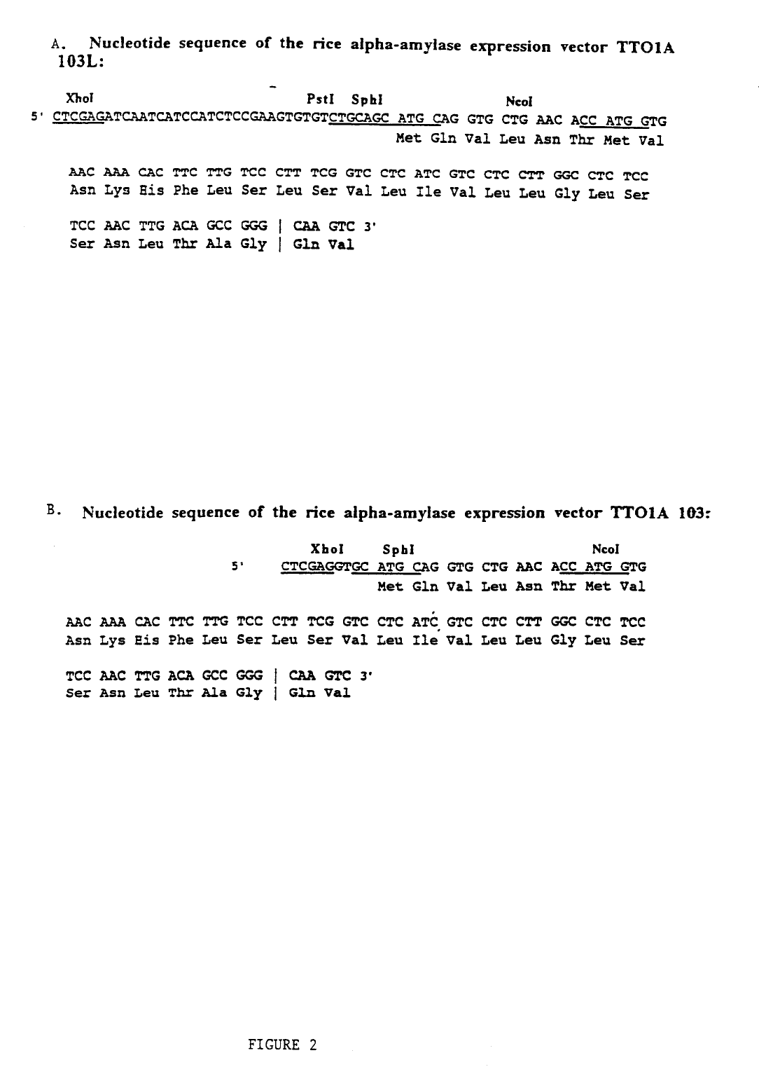

Tobamoviral vectors have been developed for the expression of heterologous proteins in plants. The rice α-amylase gene (OS103) was placed under the transcriptional control of a tobamovirus subgenomic promoter in TTO1A 103L, a RNA viral vector. One to two weeks after inoculation, transfected Nicotiana benthamiana plants accumulated glycosylated α-amylase to levels of at least 5% total soluble protein. The 46 kDa recombinant enzyme was purified and its structural and biological properties were analyzed. The rice α-amylase 5′ untranslated leader enhanced the production of recombinant enzyme in transfected plants. It is possible that there is synergy between the 5′ leader and 3′-untranslated region (UTR) of the recombinant tobamovirus. The highly expressed viral coat subgenomic RNA has a 5′ cap (m7GpppN) and terminates with a tRNA-like structure instead of a poly(A) tail. The 3′-UTR has two domains which contains five RNA pseudoknots. The tobacco etch viral (TEV) 5′ leader and poly(A) tail are synergistic regulators of translation in transfected plants and animal cells. In the present embodiment, a modified α-amylase cDNA was placed under the control of the TMV-U1 coat protein subgenomic promoter. The 34 bp rice α-amylase 5′ untranslated leader can help to enhance the initiation of translation, the stability of viral sequences, and the synthesis of subgenomic RNA. There was at least a one hundred fold increase in the accumulation of α-amylase in plants transfected with constructs containing the 34 bp rice α-amylase 5′ untranslated leader (5′-G ATC AAT CAT CCA TCT CCG AAG TGT GTC TGC AGC-3′ (SEQ ID NO: 17), see FIG. 2A) compared to plants transfected with TTO1A 103, a construct that contains only a 5 bp leader (5′-GG TGC-3′, see FIG. 2B).

### Example 2

Construction of Cytoplasmic Expression Vector Containing the Rice α-Amylase 5′ untranslated leader

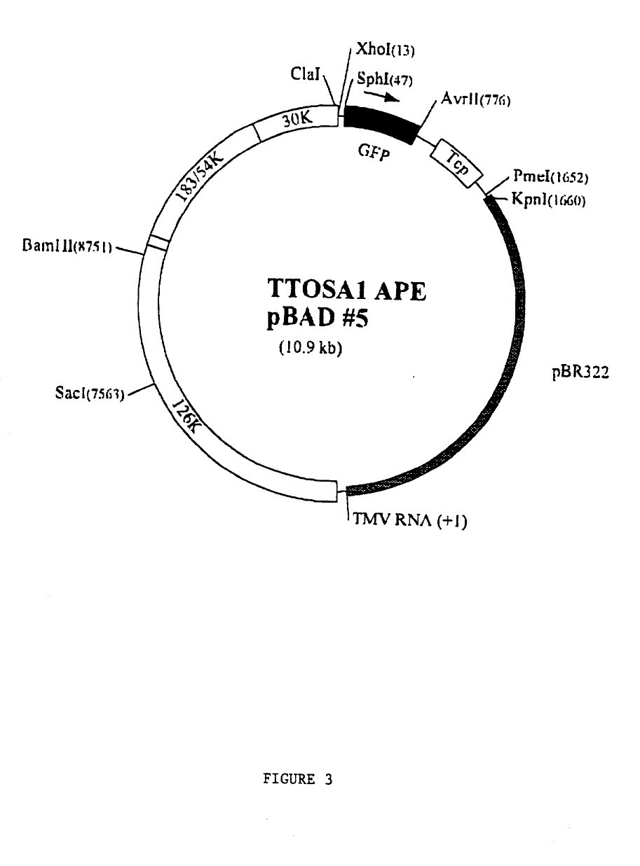

TTOSA1 APE pBAD was designed to express GFP in the cytoplasm. Using PCR mutagenesis, the SphI site in the 126K replicase open reading frame (ORF) of TTO1A was removed using oligonucleotide 5′-CGT CCA GGT TGG GCA TAC AGC AGT GTA CAT ATG C-3′ (SEQ ID NO: 18) and a unique PmeI site was inserted at the 3′ end of tomato mosaic virus cDNA (fruit necrosis train F; ToMV-F) using oligonucleotide 5′-CGG GGT ACC GTT TAA ACT GGG CCC CAA CCG GGG GTT CCG GG-3′ (SEQ ID NO: 19). A 1.4 Kb XhoI, AvrII fragment from TTO1A 103L containing the rice α-amylase OS103 cDNA (O'Neill et al., Mol. Gen. Genet. 221:235-244 (1990)) was inserted, creating plasmid TTOSA1 APE 103L. A unique SphI site (start codon) and a unique AvrII site (adjacent to the stop codon) was inserted in the jellyfish Aequorea victoria GFP cDNA by PCR mutagenesis using oligonucleotides GFP MIS 5′-TAA GCA TGC TGA AAG GAG AAG AAC TTT TCA CTG GAG TT-3′ (upstream) (SEQ ID NO: 20) and GFP K238 5′-TAC CTA GGA GAT ATC CTT GTA TAG TTC ATC CAT GCC ATG TGT-3′ (downstream) (SEQ ID NO: 21, subcloned into TTOSA1 APE 103L, creating plasmid TTOSA1 APE pBAD #5 (FIG. 3).

### Example 3

Construction of Secretion Vector Containing the Rice Alpha-Amylase 5′ Untranslated Leader

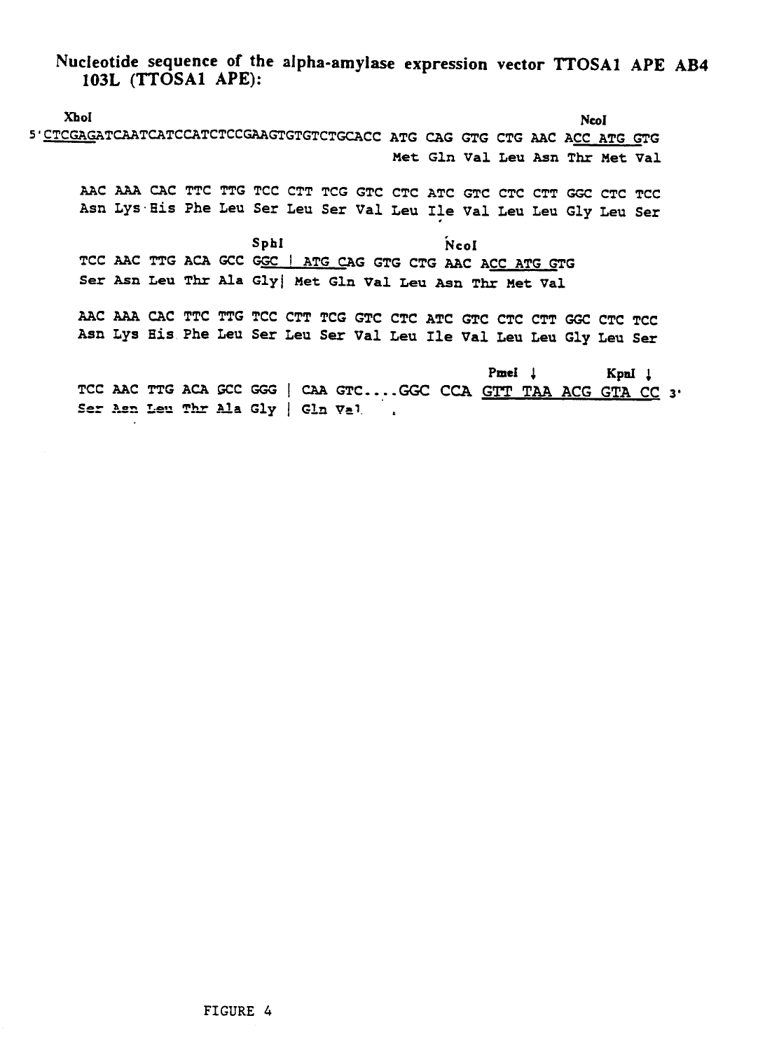

Using polymerase chain reaction (PCR) mutagenesis, the SphI site in the 126K replicase open reading frame (ORF) of TTO1A was removed using oligonucleotide 5′ CGT CCA GGT TGG GCA TAC AGC AGT GTA CAT ATG C 3′ (SEQ ID NO: 22), and a unique PmeI site was inserted at the 3′ end of tomato mosaic virus cDNA (ToMV) using oligonucleotide 5′-CGG GGT ACC GTT TAA ACT GGG CCC CAA CCG GGG GTT CCG GG-3′ (SEQ ID NO: 23). Unique XhoI, AvrII sites were inserted into the rice α-amylase OS103 cDNA by PCR mutagenesis using oligonucleotides: 5′-CTC TCG AGA TCA ATC ATC CAT CTC CGA AGT GTG TCT GC-3′ (upstream) (SEQ ID NO: 24) and 5′ TCC CTA GGT CAG ATT TTC TCC CAG ATT GCG TAG C 3′ (downstream) (SEQ ID NO: 25) and subcloned into the SphI, PmeI modified tobamoviral vector, creating plasmid TTOSA1 APE 103L. In order to clone the 5′ untranslated leader adjacent to a modified α-amylase signal peptide ORF, we utilized a plasmid, TTOAB4, that contained a unique SphI site that was introduced into the rice α-amylase signal peptide ORF of OS103 by PCR mutagenesis using oligonucleotides 5′-GCC TCG AGT GCA CCA TGC AGG TGC TGA ACA CCA TGG TG-3′ (upstream) (SEQ ID NO: 26) and 5′-GAG CAT GCC GGC TGT CAA GTT GGA GGA GAG GCC-3′ (downstream) (SEQ ID NO: 27). An NcoI fragment from TTO1A 103L containing part of the TMV-U1 30K ORF, 5′ untranslated leader, and six codons of the rice α-amylase was subcloned into TTOAB4, creating plasmid TTO1/TTOAB4. Finally, the SphI, KpnII α-amylase ORF/ToMV 3′ end containing fragment from TTOSA1 APE 103L was subcloned into TTO1/TTOAB4 creating plasmid TTOSA1 APE AB4 103L (TTOSA1 APE) (FIG. 4) (SEQ ID NOs: 7 and 8).

### Example 4

Construction of Secretion Vector Containing the Rice Alpha-Amylase 5′ Untranslated Leader and Non-Hodgkin's Lymphoma (NHL) Single Chain Antibody cDNA

Autonomously replicating RNA viral vectors were developed for the production and secretion of heterologous proteins in plants. These constructs were derived from hybrid fusions of two tobamoviruses and contained additional subgenomic promoters for expression of foreign genes. A sequence encoding a modified rice α-amylase signal peptide (OS103) was fused to a single chain Fv (scFv) open reading frame in the tobamoviral vector TTOSA1 APE (McCormick et al., Proc. Natl. Acad. Sci. USA 96:703-708 (1999)).

Construction of the Single Chain Antibody Expression Vector NHL

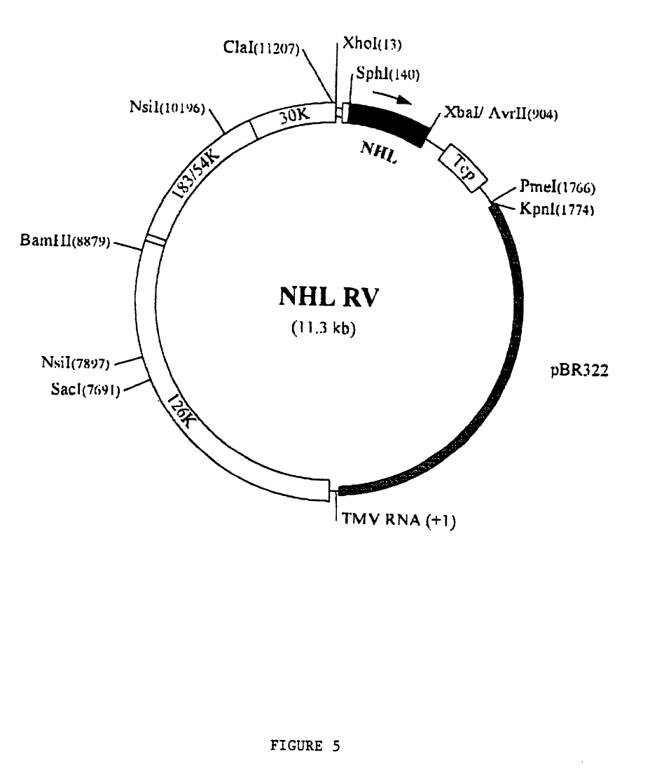

PCR primers specific for murine 38C13 sequences (GenBank accession nos. X14096-X14099) were used to amplify the 38C13 scFv coding sequence. 38C13 scFv insert was then cloned in-frame with the sequence encoding a rice -amylase signal peptide into TTOSA1 APE AB4 103L, a modified TTO1A vector containing a hybrid fusion of TMV and tomato mosaic virus. The resulting plasmid was named NHL RV (FIG. 5).

Expression, and Purification of 38C13 scFv from Transfected N. benthamiana

Infectious RNA transcripts were made in vitro and directly applied to plants. High-level expression and accumulation of the single chain antibody occurred within ten days post inoculation. The interstitial fluid containing the scFv was isolated using vacuum infiltration and the secreted protein was purified to homogeneity by affinity chromatography. Infected N. benthamiana plants contained high levels of secreted scFv protein in the extracellular compartment. The material reacted with an anti-idiotype antibody by Western blotting, ELISA, and affinity chromatography, suggesting that the plant-produced 38C13 scFv protein was properly folded in solution. Mice vaccinated with the affinity-purified 38C13 scFv generated>10 μg/ml anti-idiotype immunoglobulins. These mice were protected from challenge by a lethal dose of the syngeneic 38C13 tumor, similar to mice immunized with the native 38C13 IgM-keyhole limpet hemocyanin conjugate vaccine. This rapid production system for generating tumor-specific protein vaccines may provide a viable strategy for the treatment of non-Hodgkin's lymphoma.

### Example 5

Construction of an Artificial Leader Using a Modified TMV Coat ORF

During replication of the tobamoviral vectors, a small amount of negative strand RNA is synthesized. The native subgenomic promoter is located on the minus strand and controls the expression of foreign genes. Although deletion analysis of sequences surrounding the TMV coat protein transcriptional start site revealed that the major portion of the subgenomic promoter was upstream of the coat AUG, a small portion of the promoter may reside downstream of the start codon. In order to address this issue, an artificial leader was constructed by mutating the TMV coat protein start codon ATG to AGA by site-directed mutagenesis. Foreign gene inserted downstream of the artificial leader sequence (5′-TCTTACAGTATCACTACTCCATCTCAGTTCGTGTTCTTGTCA-3′) (SEQ ID NO: 28) at several unique cloning sites, showed increased genetic stability and led to a higher level of when compared with virus constructs lacking the leader sequence.

### Example 6

Construction of Secretion Vector Containing an Artificial Leader and a Human BA46 Gene

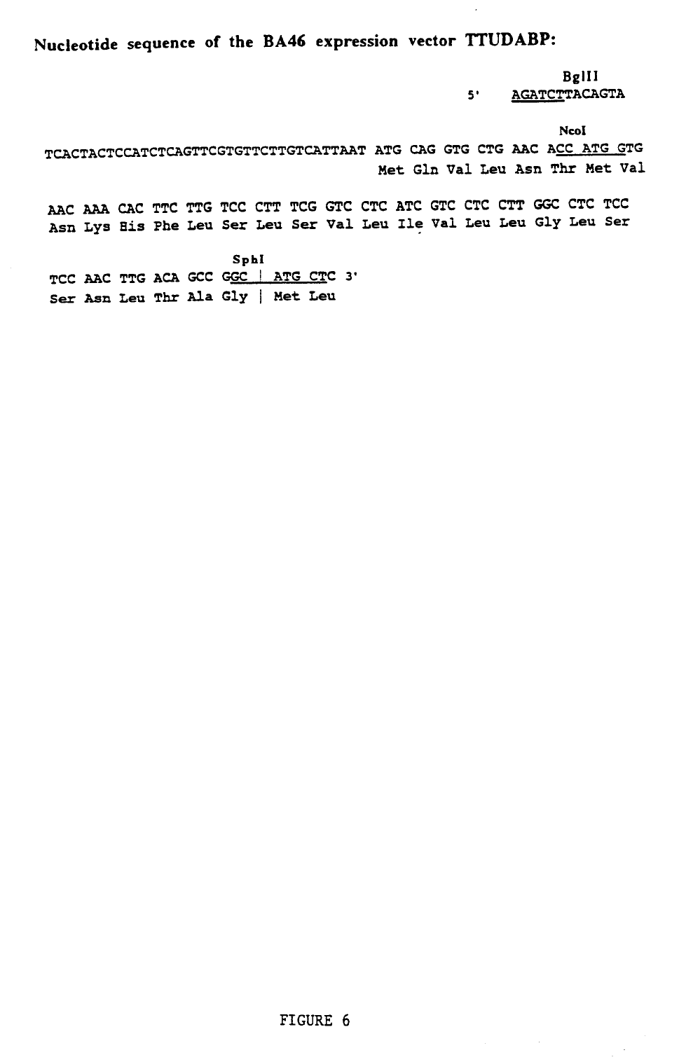

In several cloning steps a secretion vector was constructed that contains a hybrid virus, TTU51, consisting of TMV-U1 and tobacco mild green mosaic virus (TMGMV; U5 strain) and the sequence encoding a modified rice α-amylase signal peptide. In this plasmid the SphI site in the 126K replicase open reading frame was removed using oligonucleotide 5′-CGT CCA GGT TGG GCA TAC AGC AGT GTA CAT ATG C-3′ (SEQ ID NO: 29), and a 1-Kb AvrII-KpnI TMGMV 3′ end from TTU51 was attached. Unique SphI, AvrII sites were inserted into human BA46 cDNA (Couto et al., DNA Cell Biology 15:281-286 (1996)) by polymerase chain reaction (PCR) mutagenesis using oligonucleotides: 5′-CTC GAG GCA TGC TCC TGG ATA TCT GTT CCA AAA ACC-3′ (upstream) (SEQ ID NO: 30) and 5′ GAC CGG TCC TAG GTT AAC AGC CCA GCA GCT CCA GGC GCA GGG C 3′ (downstream) (SEQ ID NO: 31) and subcloned into the tobamoviral secretion vector, creating plasmid TTUDABP (FIG. 6). Infectious RNA transcripts were made in vitro and directly applied to plants. One week after transfection, recombinant human BA46 was detected in systemically infected tissue using an anti-BA46 antibody.

### Example 7

Construction of β-Globin Expression Vector

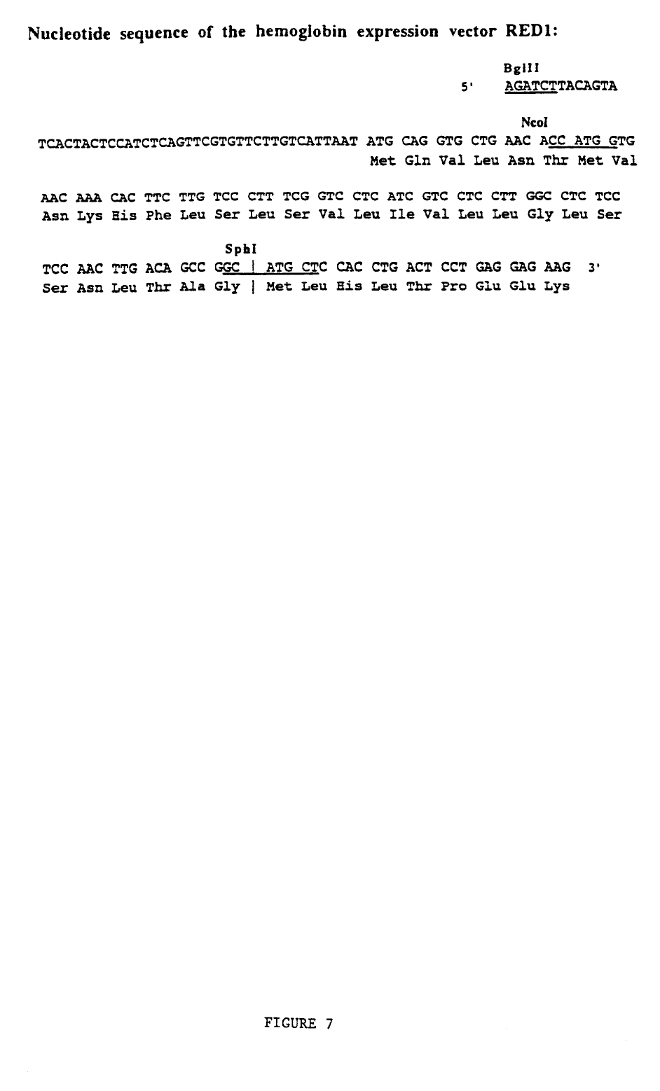

The hemoglobin expression vector, RED1 (FIG. 7), was constructed in several subcloning steps. A unique SphI site was inserted in the start codon for the human β-globin and an XbaI site was placed downstream of the stop codon by polymerase chain reaction (PCR) mutagenesis by using oligonucleotides 5′ CAC TCG AGA GCA TGC TGC ACC TGA CTC CTG AGG AGA AG 3′ (upstream) (SEQ ID NO: 32) and 5′-CGT CTA GAT TAG TGA TAC TTG TGG GCC AGC GCA TTA GC-3′ (downstream) (SEQ ID NO: 33). The 452 bp SphI-XbaI hemoglobin fragment was subcloned into the SphI-AvrII site of a modified tobamoviral vector, TTU51D. This construct consisits of a 1020 bp fragment from the tobacco mild green mosaic virus (TMGMV; U5 strain) containing the viral subgenomic promoter, coat protein gene, and the 3′ end that was isolated by PCR using TMGMV primers 5′-GGC TGT GAA ACT CGA AAA GGT TCC GG-3′ (upstream) (SEQ ID NO: 34) and 5′-CGG GGT ACC TGG GCC GCT ACC GGC GGT TAG GGG AGG-3′ (downstream) (SEQ ID NO: 35). In this vector, an artificial 40 bp 5′ untranslated coat protein leader was fused to a hybrid cDNA encoding rice α-amylase signal peptide and human β-globin. The heterologous gene was under the control of the tobacco mosaic virus (TMV-U1) coat protein subgenomic promoter. Infectious RNA transcripts were made in vitro and directly applied to plants. One week after transfection, recombinant human β-globin was detected in systemically infected tissue using an anti-hemoglobin antibody.

### Example 8

cDNA Library Construction in a Recombinant Viral Nuclei Acid Vector

cDNA libraries can be constructed or obtained from a variety of private or public sources such as the Arabidopsis Biological Resource Center (ABRC). The cDNA libraries can be digested with appropriate restriction enzymes and the inserts can be modified by adding linker adapters with cohesive ends, and directly cloned into recombinant viral nucleic acid vectors containing non-native 5′ untranslated leader sequences. Bacterial cells can be transformed with the viral based cDNA library. DNA that is isolated from the cells can be used to make infectious RNA that is directly applied to plants. The viral constructs causing changes in the phenotype or biochemical properties of the transfected plants can be chararcterized by nucleic acid sequencing. Selected leaf disc from the transfected plants can be taken for biochemical analysis such as MALDI-TOF. A recombinant viral nucleic acid expression vector library containing non-native 5′ untranslated leaders would be especially useful in detecting tranfected plants that are over-expressing foreign proteins.

### Example 9

Use of Inserted Non-Native Sequences to Enhance the Expression of Foreign Genes in Transfected Plants

Insertion of foreign gene sequences into virus expression vectors can result in arrangements of sequences that interfere with normal virus function and thereby, establish a selection landscape that favors the genetic deletion of the foreign sequence. Such events are adverse to the use of such expression vectors to stably express gene sequences systemically in plants. A method that would allow sequences to be identified that may insulate functional virus sequences from the potential adverse effects of insertion of foreign gene sequences would greatly augment the expression potential of virus expression vectors. In addition, identification of such “insulating” sequences that simultaneously enhanced the translation of the foreign gene product or the stability of the mRNA encoding the foreign gene would be quite helpful. The example below demonstrates how libraries of random sequences can be introduced into virus vectors flanking foreign gene sequences. Upon analysis, a subset of introduced sequences allowed a foreign gene sequence that was previously prone to genetic deletion to remain stabily in the virus vectors upon serial passage. The use of undefined sequences to enhance the stability of foreign gene sequences can be extrapolated to the use of undefined sequences to enhance the translation of foreign genes and the stability of coding mRNAs by those skilled in the art.

Undefined sequences can also be used to enhance and extend the expression of foreign genes in a viral vector. To test this hypothesis random sequences of N20 were cloned in-between the TMV subgenomic promoter and the gene sequence for either human growth hormone (hGH) or a ubiquitin-hGH fusion gene. In this experiment the site of random nucleotide insertion was following a PacI (underlined) restriction enzyme site in the virus vector. This sequence is known as a leader sequence and has been derived from the native leader and coding region from the native TMV U1 coat protein gene. In this leader, the normal coat protein ATG has been mutated to a Aga sequence (underlined in GTTTTAAATAgaTCTTACAGTATCACTACTCCATCTCAGTTCGTGTTCTTGT CATTAATTAA ATG . . . (hGH GENE)) (SEQ ID NO: 36). A particular subset of this leader sequence (TCTTACAGTATCACTACTCCATCTCAGTTCGTGTTCTTGTCA) (SEQ ID NO: 28) has been known to increase genetic stability and gene expression when compared with virus construct lacking the leader sequence. The start site of subgenomic RNA synthesis is found at the GTTTT . . . An oligonucleotide RL-1 (GTTTTAAATAGATCTTAC N(20)TTAATTAAGGCC ) (SEQ ID NO: 37) was used with a primer homologous to the NcoI/ApaI region of the TMV genome to amplify a portion of the TMV movement protein. The population of sequences were cloned into the ApaI and PacI sites of the p30B hGH vector. Vectors containing the undefined sequences leading the hGH genes were transcribed and inoculated onto Nicotiana benthamiana plants. Fourteen days post inoculation, systemic leaves were ground and the plant extracts were inoculated onto a second set of plants. Following the onset of virus symptoms in the second set of plants, Western blot analysis was used to detect if hGH or Ubiq-hGH fusions were present in the serially inocuated plants. Several variants containing novel sequences in the non-translated leader sequence were associated with viruses that expressed higher than control levels of hGH or Ubiquitin hGH fusion proteins in plants inoculated with in vitro synthesized transcripts or upon serial passage of virus. The sequence surrounding the leader was determined and compared with that of the control virus vectors (SEQ ID NOs: 38-49).

| | |
|---|---|
| p30B #5 HGH | GTTTTAAATAGATCTTAC--TATAACATGAATAGTCATCG |
| p30B #5 HGH | GTTTTAAATAGATCTTAC--TATACCATGAATTAGTACCG |
| p30B #6 UbiqHGH | GTTTTAAATAGATCTTAC--ACTCGGTTGAGATAAAACTAAACTA |
| p30B #2 HGH | GTTTTAAATAGATCTTAC--TCCGACGTATAGTCACCACG |
| p30B HGH | GTTTTAAATAGATCTTAC--AGTATCACTACTCCATCTCAGTTCGTGTTCT |
| p30B UbiqHGH | GTTTTAAATAGATCTTAC--AGTATCACTACTCCATCTCAGTTCGTGTTCT |
| | ***************** |
| p30B #5 HGH | ---TTAATTAAAATGGGA--- |
| p30B #5 HGH | ---TTAATTTAAAATGGGAAAAATGGCTTCTCTATTTGCCACATTTTTA |
| p30B #6 UbiqHGH | ---TTAATTAAAATGGGAAAAATGGCTCTCTTATTGGCCCCATTTTTA |
| p30B #2 HGH | ---TTAATTAAAAATGCAGATTTTCGTCAAGACTTTGACCGGG |
| p30B HGH | TGTCATTAATTAAAATGGGAAAAATGGCTTCTCTATTTGCCACATTTTTA |
| p30B UbiqHGH | TGTCATTAATTAAAATGCAGATTTTCGTCAAGACTTTGACCGGT |
| | ************ |

*indicates sequences that are identical in all viruses.
--indicates end of defined primer and start of N(20) region of the oligonucleotide that was introduced during PCR amplification.

The result was that undefined leader constructs transcribed were passageable as virus. The nature of the random leaders indicates that each are unique and that multiple solutions are readily available to solve RNA based stability problems. Likewise, such random sequence introductions could also increase the translational efficiency.

In order to select for undefined sequences that may increase the translational efficiency of foreign genes or increases the stability of the mRNA encoding the foreign gene derived from a virus expression vector, a selectable marker could be used to discover which of the undefined sequences yield the desired function. The amount of the GFP protein correlates with the level of fluorescence seen under long wave UV light and the amount of herbicide resistance gene product correlates with survival of plant cells or plants upon treatment with the herbicide. Therefore introduction of undefined sequences surrounding the GFP or herbicide resistance genes and then screening for individual viruses that either express the greatest level of fluorescence or cells that survive the highest amount of herbicide. In this manner the cells with the viruses with the highest foreign gene activity would be then purified and characterized by sequencing and more thorough analysis such as Northern and Western blotting to access the stability of the mRNA and the abundance of the foreign gene of interest.

### Example 10

Use of the Untranslated Non-Native 5′ Sequence to Enhance the Ratio of Coat Protein Fusion Protein/Coat Protein Production

U.S. Pat. No. 5,618,699 issued to Hamamoto et al. describes virion particles comprising a TMV coat protein and a fusion protein of the TMV coat protein and a foreign protein Such virion particles are produced by inoculating a plant with a viral vector comprising a foreign gene linked downstream of a TMV coat protein via a nucleotide sequence which occassionally causes readthrough. Such a “leaky stop codon” sequence results in mostly upstream coat protein gene expression, but occasionally results in readthrough expression of both the upstream coat protein and the downstream foreign protein. These coat proteins and coat protein fusion proteins will self-assemble around the vector nucleic acid, resulting in a virion particle having a coat protein subunits interspersed with coat protein fusion proteins. Because of steric hindrance, it is preferable to be able to modulate the level of coat protein fusion proteins interspersed with the coat proteins. The leaky stop codon thus useful, as it only allows readthrough transcription from the coat protein gene into the foreign protein gene a small percentage of the time.

An alternative way of modulating ratio of coat protein expression relative to coat protein fusion protein expression, is to construct a viral vector comprising both a coat protein coding sequence and a coat protein fusion protein coding sequence, with each having its own subgenomic promoter, and with a 5′ untranslated, non-native sequence of the present invention operably placed upstream of the coat protein. In this manner, the ratio of the production of coat protein fusion gene vs. that of the coat protein are increased.

Although the foregoing invention has been described in some detail by way of illustration and example for purposes of clarity of understanding, it will be obvious that certain changes and modifications may be practiced within the scope of the appended claims.

All publications, patents, patent applications are herein incorporated by reference in their entirety to the same extent as if each individual publication, patent, or patent application was specifically and individually indicated to be incorporated by reference in its entirety.

We claim:

1. A recombinant viral nucleic acid comprising:
(a) a first sequence which comprises a promoter and a non-native 5′-untranslated sequence, wherein said non-native 5′-untranslated sequence comprises an untranslated leader sequence and
(b) a second sequence which is downstream of and operatively linked to said first sequence, wherein the amount of RNA or protein produced from said second sequence is increased compared to the amount produced in the absence of said non-native sequence; wherein said recombinant viral nucleic acid comprises less than an infective viral genome.

2. The recombinant viral nucleic acid according to claim 1, wherein said recombinant viral nucleic acid is derived from an RNA plant virus.

3. The recombinant viral nucleic acid according to claim 1, wherein said recombinant viral nucleic acid native to a single-stranded, positive sense RNA plant virus.

4. The recombinant viral nucleic acid according to claim 1, wherein said recombinant viral nucleic acid is derived from an animal virus.

5. The recombinant viral nucleic acid according to claim 1, wherein said recombinant viral nucleic acid is derived from a bacterial virus.

6. The recombinant viral nucleic acid according to claim 1 wherein said non-native 5′-untranslated sequence is obtained by in vitro mutagenesis, recombination, or a combination thereof.

7. The recombinant viral nucleic acid according to claim 1 wherein said non-native 5′-untranslated sequence is constructed by moving the ATG start codon downstream to a new site, thus creating an artificial leader sequence.

8. The recombinant viral nucleic acid according to claim 1 wherein said second sequence comprises a non-native coding sequence.

9. The recombinant viral nucleic acid according to claim 8 wherein said non-native coding sequence encodes a fusion protein between a coat protein and a non-native protein or polypeptide.

10. The recombinant viral nucleic acid according to claim 8 wherein said non-native coding sequence encodes a product selected from the group consisting of enzymes, antibodies, hormones, pharmaceuticals, vaccines, pigments, and anti-microbial polypeptides.

11. A vector comprising the recombinant viral nucleic acid according to claim 1.

12. The vector according to claim 11 which is a plasmid.

13. An isolated host cell transformed with the recombinant viral nucleic acid according to claim 1.

14. The recombinant viral nucleic acid according to claim 1, wherein said first or second sequence further comprising a promoter sequence.

15. An expression vector comprising the recombinant viral nucleic acid according to claim 14.

16. The expression vector according to claim 15 which is a plasmid.

17. An isolated host cell transformed with the recombinant viral nucleic acid according to claim 14.

18. A recombinant viral nucleic acid comprising a non-native sequence inserted in any nucleotide position 5′ to the initiation codon of said recombinant viral nucleic acid, wherein the amount of RNA or protein produced from said recombinant viral nucleic acid is increased compared to the amount produced in the absence of said non-native sequence, wherein said recombinant viral nucleic acid comprises less than an infective viral genome, wherein said non-native sequence comprises an untranslated leader sequence.

19. The recombinant viral nucleic acid according to claim 18, wherein said recombinant viral nucleic acid is derived from an RNA plant virus.

20. The recombinant viral nucleic acid according to claim 18, wherein said recombinant viral nucleic acid is derived from a single stranded, positive sense RNA plant virus.

21. The recombinant viral nucleic acid according to claim 18, wherein said recombinant viral nucleic acid is derived from an animal virus.

22. The recombinant viral nucleic acid according to claim 18, wherein said recombinant viral nucleic acid is derived from a bacterial virus.

23. The recombinant viral nucleic acid according to claim 18 wherein said recombinant viral nucleic acid comprises a non-native coding sequence.

24. The recombinant viral nucleic acid according to claim 23 wherein said non-native coding sequence encodes a fusion protein between a coat protein and a non-native protein or polypeptide.

25. The recombinant viral nucleic acid according to claim 23 wherein said non-native sequence encodes a product selected from the group consisting of enzymes, antibodies, hormones, pharmaceuticals, vaccines, pigments, and anti-microbial polypeptides.

26. A vector comprising the recombinant viral nucleic acid according to claim 18.

27. The vector of claim 26 which is a plasmid.

28. An isolated host cell transformed with the recombinant viral nucleic acid according to claim 18.

29. The recombinant viral nucleic acid according to claim 18, wherein said recombinant viral nucleic acid further comprises a promoter sequence.

30. An expression vector comprising claim 29.

31. The expression vector according to claim 30 which is a plasmid.

32. An isolated host cell transformed with the recombinant viral nucleic acid according to claim 29.

33. A method for enhancing the production of a protein in a host comprising the steps of expressing in said host a recombinant viral nucleic acid comprising:
(a) a first sequence which comprises a non-native 5′-untranslated sequence, and
(b) a second sequence which is downstream of and operatively linked to said first sequence, wherein said second sequence comprises a coding sequence encoding said protein.

34. The method according to claim 33 wherein said protein is a fusion protein with a coat protein.

35. A method for enhancing the production of a protein in a host comprising the steps of expressing in said host a recombinant viral nucleic acid comprising:
(a) a non-native sequence inserted in any nucleotide position 5′ to the initiation codon of said recombinant viral nucleic acid and a coding sequence encoding said protein.

36. The method according to claim 35 wherein said protein is a fusion protein with a coat protein.


SEQUENCE LISTING (49 sequences)

SEQ ID NO: 1 - DNA, 117 bp, Tobacco mosaic virus
  CDS (49)...(117)
gttttaaatacgctcgagatcaatccatctccgaagtgtgtctgcagcatgcaggtgctgaacaccatggtgaacaaacacttcttgtccctttcggtcctcatcgtcctcatcgtc

SEQ ID NO: 2 - protein, 23 aa, Tobacco mosaic virus
MQVLNTMVNKHFLSLSVLIVLLG

SEQ ID NO: 3 - DNA, 138 bp, Tobacco mosaic virus
ctcgagatcaatcatccatctccgaagtgtgtctgcagcatgcaggtgctgaacaccatggtgaacaaacacttcttgtccctttcggtcctcatcgtcctccttggcctctcctccaacttgacagccgggcaagtc

SEQ ID NO: 4 - protein, 33 aa, Tobacco mosaic virus
MQVLNTMVNKHFLSLSVLIVLLGLSSNLTAGQV

SEQ ID NO: 5 - DNA, 109 bp, Tobacco mosaic virus
ctcgaggtgcatgcaggtgctgaacaccatggtgaacaaacacttcttgtccctttcggtcctcatcgtcctccttggcctctcctccaacttgacagccgggcaagtc

SEQ ID NO: 6 - protein, 31 aa, Tobacco mosaic virus
MQVLNTMNKHLSLSVLIVLLGLSSNLTAGQV

SEQ ID NO: 7 - DNA, 259 bp, Tobacco mosaic virus
ctcgagatcaatcatccatctccgaagtgtgtctgcaccatgcaggtgctgaacaccatggtgaacaacacttcttgtccctttcggtcctcatcgtcctccttggcctctcctccaacttgacagccggcatgcaggtgctgaacaccatggtgaacaaacacttcttgtccctttttgtccctttcggtcctcatcgtcctccttggcctctcctccaacttgacagccggcaagtcggcccagtttaaacggtacc

SEQ ID NO: 8 - protein, 60 aa, Tobacco mosaiv virus
MQVNTMVNKHFLSLSVLIVLLGLSSNLTAGMQVLNTMVNKHFLSVLIVLLGLSSLTAGQV

SEQ ID NO: 9 - DNA, 170 bp, Tobacco mosaic virus
  CDS (51)...(170)
agatcttacagtatcactactccatctcagttcgtgttcttgtcattaatatgcaggtgctgaacaccatggtgaacaaacacttcttgtccctttcggtcctcatcgtcctccttggcctctcctccaacttgacagccggcatgctccacctgactcctgaggagaag

SEQ ID NO: 10 - protein, 40 aa, Tobacco mosaic virus
MQVLNTMVNKHFLSLSVLIVLLGLSSNLTAGMLHLTPEEK

SEQ ID NO: 11 - DNA, 149 bp, Tobacco mosaic virus
  CDS (51)...(146)
agatcttacagtatcactactccatctcagttcgtgttcttgtcattaatatgcaggtgctgaacaccatggtgaacaaacacttcttgtccctttcggtcctcatcgtcctccttggcctctcctccaacttgacagccggcatgctc

SEQ ID NO: 12 - protein, 32 aa, Tobacco mosaic virus
MQVLNTMVNKHFLSLSVLIVLLGLSSNLTAGM

SEQ ID NO: 13 - DNA, 38 bp, Tobacco mosaic virus
gcctcgagtgcaccatgcaggtgctgaacaccatggtg

SEQ ID NO: 14 - DNA, 46 bp, Tobacco mosaic virus
tccctaggtcagattttctcccagattttctcccagattgcgtagc

SEQ ID NO: 15 - DNA, 38 bp, Tobacco mosaic virus
ctctcgagatcaatcatccatctccgaagtgtgtctgc

SEQ ID NO: 16 - DNA, 34 bp, Tobacco mosaic virus
tccctaggtcagattttctcccagattgcgtagc

SEQ ID NO: 17 - DNA, 34 bp, Nicotiana  benthamiana
gatcaatcatccatctccgaagtgtgtctgcagc

SEQ ID NO: 18 - DNA, 34 bp, Nicotiana benthamiana
cgtccaggttgggcatacagcagtgtacatatgc

SEQ ID NO: 19 - DNA, 41 bp, Nicotiana benthamiana
cggggtaccgtttaaactgggccccaaccgggggttccggg

SEQ ID NO: 20 - DNA, 38 bp, Nicotiana benthamiana
taagcatgctgaaaggagaagaacttttcactggagtt

SEQ ID NO: 21 - DNA, 43 bp, Nicotiana benthamiana
ctacctaggagatatccttgtatagttcatccatgccatgtgt

SEQ ID NO: 22 - DNA, 34 bp, Nicotiana benthamiana; identical to SEQ ID NO: 18

SEQ ID NO: 23 - DNA, 41 bp, Nicotiana benthamaiana; identical to SEQ ID NO: 19

SEQ ID NO: 24 - DNA, 35 bp, Tobacco mosaic virus
tcgagatcaatcatccatctccgaagtgtgtctgc

SEQ ID NO: 25 - DNA, 34 bp, Tobacco mosaic virus; identical to SEQ ID NO: 16

SEQ ID NO: 26 - DNA, 38 bp, Tobacco mosaic virus; identical to SEQ ID NO: 13

SEQ ID NO: 27 - DNA, 33 bp, Tobacco mosaic virus
gagcatgccggctgtcaagttggaggagaggcc

SEQ ID NO: 28 - DNA, 42 bp, Tobacco mosaic virus
tcttacagtatcactactccatctcagttcgtgttcttgtca

SEQ ID NO: 29 - DNA, 34 bp, Tobacco mosaic virus; identical to SEQ ID NO: 18

SEQ ID NO: 30 - DNA, 36 bp, Tobacco mosaic virus
ctcgaggcatgctcctggatatctgttccaaaaacc

SEQ ID NO: 31 - DNA, 43 bp, Tobacco mosaic virus
gaccggtcctaggttaacagcccagcagctccaggcgcagggc

SEQ ID NO: 32 - DNA, 38 bp, Tobacco mosaic virus
cactcgagagcatgctgcacctgactcctgaggagaag

SEQ ID NO: 33 - DNA, 38 bp, Tobacco mosaic virus
cgtctagattagtgatacttgtgggccagcgcattagc

SEQ ID NO: 34 - DNA, 26 bp, Tobacco mosaic virus
ggctgtgaaactcgaaaaggttccgg

SEQ ID NO: 35 - DNA, 36 bp, Tobacco mosaic virus
cggggtacctgggccgctaccggcggttaggggagg

SEQ ID NO: 36 - DNA, 66 bp, Tobacco mosaic virus
cgttttaaatagatcttacagtatcactactccatctcagttcgtgttcttgtcattaattaaatg

SEQ ID NO: 37 - DNA, 31 bp, Nicotiana benthamiana
  misc_feature (19)...(19): n = a, t, c, or g
gttttaaatagatcttacnttaattaaggcc

SEQ ID NO: 38 - DNA, 38 bp, Nicotiana benthamiana
gttttaaatagatcttactataacatgaatagtcatcg

SEQ ID NO: 39 - DNA, 38 bp, Nicotiana benthamiana
gttttaaatagatcttactataccatgaattagtaccg

SEQ ID NO: 40 - DNA, 43 bp, Nicotiana benthamiana
gttttaaatagatcttacactcggttgagataaaactaaacta

SEQ ID NO: 41 - DNA, 38 bp, Nicotiana benthamiana
gttttaaatagatcttactccgacgtatagtcaccacg

SEQ ID NO: 42 - DNA, 49 bp, Nicotiana benthamiana
gttttaaatagatcttacagtatcactactccatctcagttcgtgttct

SEQ ID NO: 43 - DNA, 49 bp, Nicotiana benthamiana; identical to SEQ ID NO: 42

SEQ ID NO: 44 - DNA, 16 bp, Nicotiana benthamiana
ttaattaaaattggga

SEQ ID NO: 45 - DNA, 46 bp, Nicotiana benthamiana
ttaatttaaaatgggaaaaatggcttctctatttgccacattttta

SEQ ID NO: 46 - DNA, 45 bp, Nicotiana benthamiana
ttaattaaaatgggaaaaatggctctcttattggccccattttta

SEQ ID NO: 47 - DNA, 40 bp, Nicotiana benthamiana
ttaattaaaaatgcagattttcgtcaagactttgaccggg

SEQ ID NO: 48 - DNA, 50 bp, Nicotiana benthamiana
tgtcattaattaaaatgggaaaaatggcttctctatttgccacattttta

SEQ ID NO: 49 - DNA, 44 bp, Nicotiana benthamiana
tgtcattaattaaaatgcagattttcgtcaagactttgaccggt